SEQUENCE LISTING (9 sequences)

SEQ ID NO: 1 - protein, 241 aa, synthetic construct
  REGION 1..241: note = Synthesized
EIVMTQSPATLSVSPGERATLSCRASQSVSRNLAWYQQKPGQAPRLLIYGASTRATGIPARFSGSGSGTEFTLTIGSLQSEDFAVYYCQQYKTWPRTFGQGTNVEIKGGGGSGGGGSGGGGSQVQLQQWGAGLLKPSETLSLTCAVFGGSFSGYYWSWIRQPPGKGLEWIGEINHRGNTNDNPSLKSRVTISVDTSKNQFALKLSSVTAADTAVYYCARERGYTYGNFDHWGQGTLVTVSS

SEQ ID NO: 2 - protein, 485 aa, synthetic construct
  REGION 1..485: note = Synthesized
MALPVTALLLPLALLLHAARPEIVMTQSPATLSVSPGERATLSCRASQSVSRNLAWYQQKPGQAPRLLIYGASTRATGIPARFSGSGSGTEFTLTIGSLQSEDFAVYYCQQYKTWPRTFGQGTNVEIKGGGGSGGGGSGGGGSQVQLQQWGAGLLKPSETLSLTCAVFGGSFSGYYWSWIRQPPGKGLEWIGEINHRGNTNDNPSLKSRVTISVDTSKNQFALKLSSVTAADTAVYYCARERGYTYGNFDHWGQGTLVTVSSAKPTTTPAPRPPTPAPTIASQPLSLRPEACRPAAGGAVHTRGLDFACDIYIWAPLAGTCGVLLLSLVITKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 3 - protein, 288 aa, synthetic construct
  REGION 1..288: note = Synthesized
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFAPRCRERRRNERLRRESVRPV

SEQ ID NO: 4 - protein, 329 aa, synthetic construct
  REGION 1..329: note = Synthesized
MDPQCTMGLSNILFVMAFLLSGAAPLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGVMQKSQDNVTELYDVSISLSVSFPDVTSNMTIFCILETDKTRLLSSPFSIELEDPQPPPDHIPWITAVLPTVIICVMVFCLILWKWKKKKRPRNSYKCGTNTMEREESEQTKKREKIHIPERSDEAQRVFKSSKTSSCDKSDTCF

SEQ ID NO: 5 - protein, 254 aa, synthetic construct
  REGION 1..254: note = Synthesized
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 6 - protein, 111 aa, synthetic construct
  REGION 1..111: note = Synthesized
DIELTQSPASLAVSLGQRATISCRASESVEYYVTSLMQWYQQKPGQPPKLLIFAASNVESGVPARFSGSGSGTNFSLNIHPVDEDDVAMYFCQQSRKVPYTFGGGTKLEIK

SEQ ID NO: 7 - protein, 120 aa, synthetic construct
  REGION 1..120: note = Synthesized
EVKLQQSGPGLVTPSQSLSITCTVSGFSLSDYGVHWVRQSPGQGLEWLGVIWAGGGTNYNSALMSRKSISKDNSKSQVFLKMNSLQADDTAVYYCARDKGYSYYYSMDYWGQGTSVTVSS

SEQ ID NO: 8 - protein, 121 aa, synthetic construct
  REGION 1..121: note = Synthesized
QVQLQQWGAGLLKPSETLSLTCAVYGGSFSGYYWSWIRQSPEKGLEWIGEINHGGYVTYNPSLESRVTISVDTSKNFSLKLSSVTAADTAVYYCARDYGPGNYDWYFDLWGRGTLVTVSSV

SEQ ID NO: 9 - protein, 109 aa, synthetic construct [sequence omitted]

(19) United States
(12) Patent Application Publication
(10) Pub. No.: US 2024/0058337 A1
(43) Pub. Date: Feb. 22, 2024

(54) Managing Side Effects in T Cell Therapy

(71) Applicants: Innovative Cellular Therapeutics Holdings, Ltd., George Town (KY); Innovative Cellular Therapeutics, Inc., Rockville, MD (US)

(72) Inventors: Lei Xiao, Rockville, MD (US); Zhao Wu, Shanghai (CN); Chengfei Pu, Shanghai (CN); Zhiyuan Cao, Shanghai (CN); Dongqi Chen, Shanghai (CN); Xiaobin Lu, Rockville, MD (US); Le Tian, Rockville, MD (US)

(73) Assignees: Innovative Cellular Therapeutics Holdings, Ltd., George Town (KY); Innovative Cellular Therapeutics, Inc., Rockville, MD (US)

(21) Appl. No.: 18/450,545

(22) Filed: Aug. 16, 2023

Related U.S. Application Data
(60) Provisional application No. 63/371,984, filed on Aug. 19, 2022.

(51) Int. Cl. A61K 31/506; A61K 39/395; A61K 39/00; A61K 31/573; A61P 35/00

(52) U.S. Cl. CPC A61K 31/506; A61K 39/3955; A61K 39/4631; A61K 31/573; A61K 39/4611; A61K 39/4612; A61P 35/00

(57) ABSTRACT

The present disclosure relates to compositions and methods for reducing side effects and/or enhancing cancer treatment that use modified immune cells expressing chimeric antigen receptors (CARs) or modified T cell receptors (TCRs). The modified immune cells, such as T cells or NK cells, can express CARs or TCRs targeting solid tumor antigens, white blood cell antigens like CD19, or bispecific CARs/TCRs targeting both. The methods include administering dasatinib to reduce the side effects associated with CAR T or TCR therapy and/or enhance cancer treatment. The modified cells can co-express additional therapeutic agents like cytokines.

### CROSS REFERENCE TO RELATED PATENT APPLICATIONS

This application also claims the benefit of U.S. Provisional Application 63/371,984, filed Aug. 19, 2022, which was hereby incorporated by reference in their entirety.

### SEQUENCE LISTING INFORMATION

The Sequence Listing associated with this application is provided in XML format in lieu of a paper copy and is hereby incorporated by reference into the specification. The name of the XML file containing the Sequence Listing is 1071-0116US.xml. The XML file is 14,196 bytes, was created on Aug. 10, 2023, and is being submitted electronically via PatentCenter.

### TECHNICAL FIELD

The present disclosure relates to compositions and methods for expanding and maintaining modified cells including genetically modified cells, and uses thereof in treating diseases, including cancer.

### BACKGROUND

Cancer refers to a large group of diseases involving abnormal cell growth. Cancer can start in almost any organ or tissue of the body and spread to other parts of the body. A tumor is also caused by an abnormal growth of cells, but can be benign or malignant. A cancerous tumor is malignant and will invade other parts of the body.

In humans, there are more than one hundred types of cancer. One example is breast cancer occurring in the epithelial tissue of the breast. Since breast cancer cells lose the characteristics of normal cells, the connection between breast cancer cells is lost. Once cancer cells are exfoliated, they spread over the entire body via the blood and/or lymph systems, becoming life-threatening. Currently, breast cancer has become one of the common threats to women's physical and mental health. Although immunotherapy, such are CAR T, has been proven effective for treating some cancers, there is still a need to improve immunotherapy to treat more cancers, including those involving solid tumors, effectively.

### SUMMARY

The present disclosure relates to methods for reducing side effects and/or enhancing cancer treatment that use modified immune cells expressing chimeric antigen receptors (CARs) or modified T cell receptors (TCRs). The modified immune cells, such as T cells or NK cells, can express CARs or TCRs targeting solid tumor antigens, white blood cell (WBC) antigens like CD19, or bispecific CARs/TCRs targeting both. The cells can co-express additional therapeutic agents, such as cytokines.

To manage the one or more side effects, such as diarrhea, neurotoxicity, or cytokine release syndrome (CRS), the methods described herein involve administering dasatinib (Sprycel®) at a first dose, then escalating to a higher second dose if side effects persist after a time period. The CARs/TCRs comprise antigen-binding domains, transmembrane domains, and intracellular signaling domains with costimulatory domains. The targets include various solid tumor antigens and white blood cell antigens. The therapeutic agents encoded in the modified cells include cytokines like IL-12 and IFN-γ. Their expression can be regulated by promoters including NFAT. Alternatively, therapeutic agents, such as fusion proteins or bispecific antibodies, can be administered that target both immune cell receptors like CD3 and tumor antigens. The modified cells can also express dominant negative immune checkpoint inhibitors, for example, PD-1 to enhance activity.

Overall, the methods provide approaches to manage the side effects of CAR/TCR cell therapy while maintaining anti-tumor efficacy and enhancing immune cell function against cancer. The embodiments described herein include preparation of modified cells, delivery of therapeutic agents, alleviation of side effect, and administration of combination therapies for improved outcomes.

This Summary is not intended to identify key features or essential features of the claimed subject matter, nor is it intended to be used to limit the scope of the claimed subject matter.

### BRIEF DESCRIPTION OF THE DRAWINGS

The Detailed Description is described with reference to the accompanying figures. The use of the same reference numbers in different figures indicates similar or identical items.

FIG. 1 shows a schematic diagram of a CoupledCAR® system.

FIG. 2 shows an exemplary embodiment of a CoupledCAR® system.

FIG. 3 shows an exemplary embodiment of a CoupledCAR® system.

FIG. 4 shows an exemplary embodiment of a CoupledCAR® system.

FIG. 5 shows a schematic diagram of a mixed population of modified cells, for example CAR T cells.

FIG. 6 shows a schematic diagram of a mixed population of modified cells comprising CARs.

FIG. 7 shows a schematic diagram of a mixed population of modified cells comprising CARs.

FIGS. 8A-8B are schematic diagrams showing dasatinib inhibits the proliferation and survival of CD19 CAR T cells while having less impact on solid tumor CAR T cells in the CoupledCAR® system.

FIGS. 9A-9C show changes of signaling pathways of CAR T cells in the CoupledCAR® system.

FIG. 10 shows a schematic plot of diarrhea management.

FIG. 11 shows a schematic plot of immune effector cell-associated neurotoxicity syndrome (ICANS) management.

FIG. 12 shows a schematic plot of CRS management.

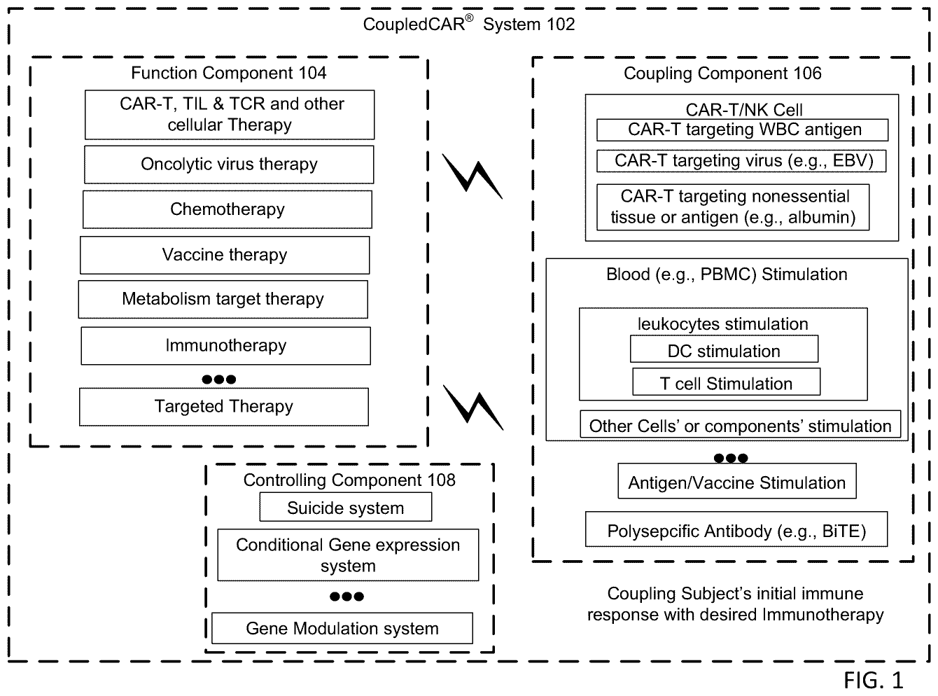

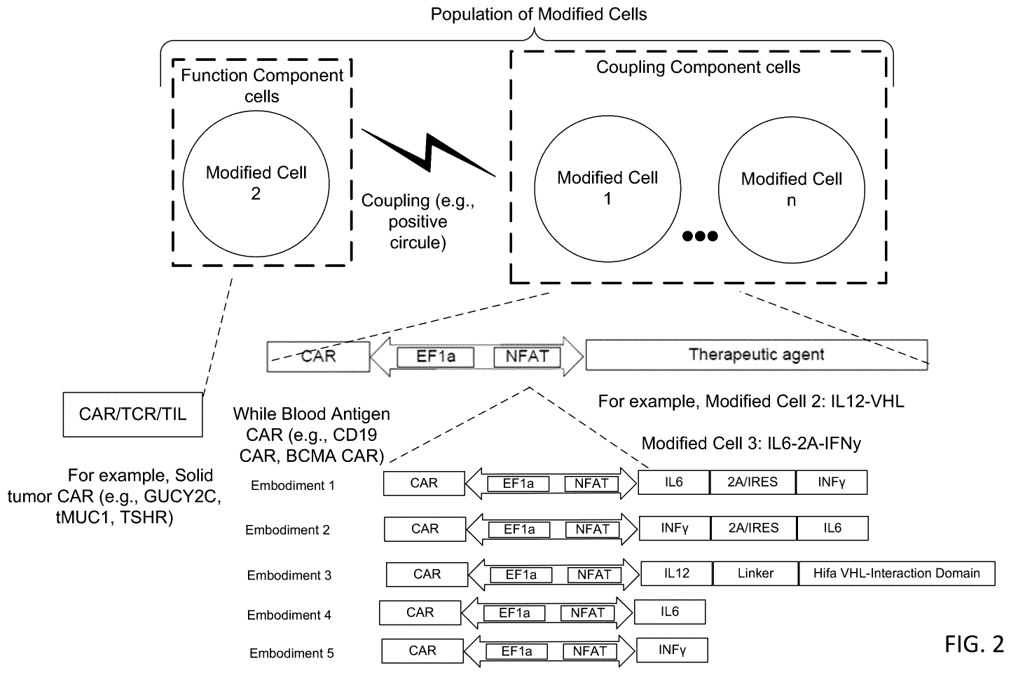

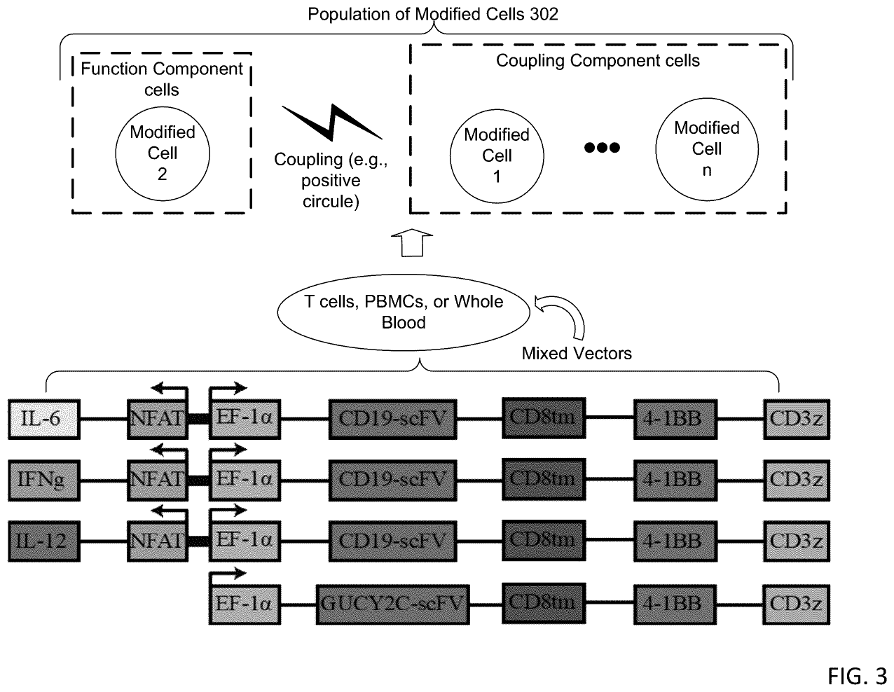

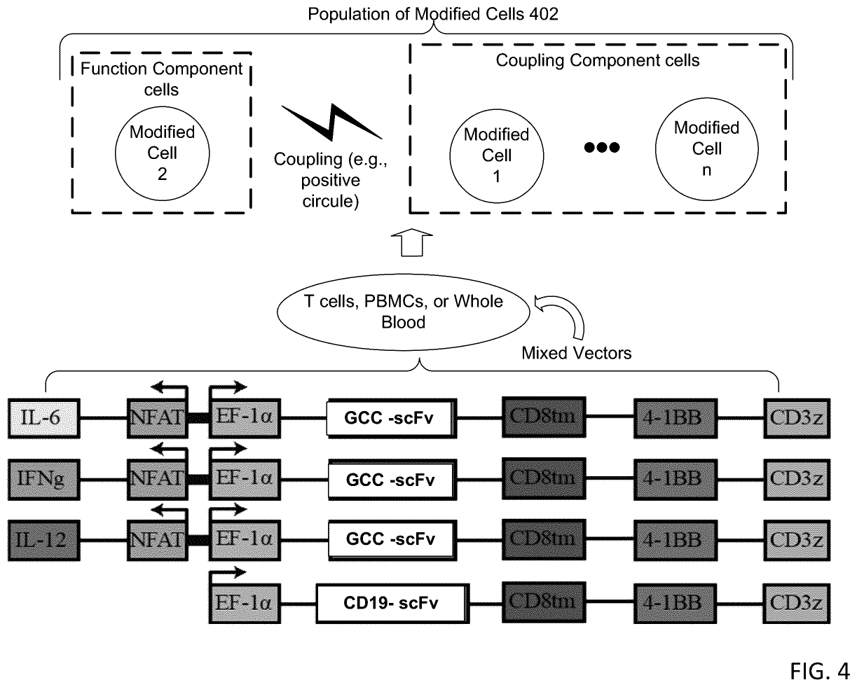

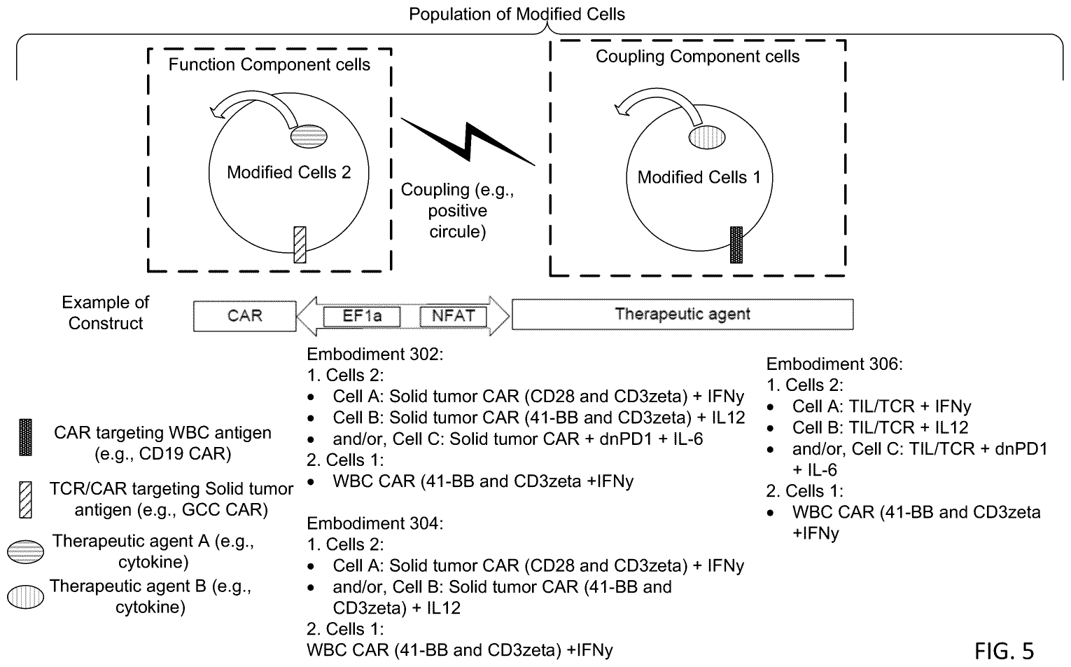

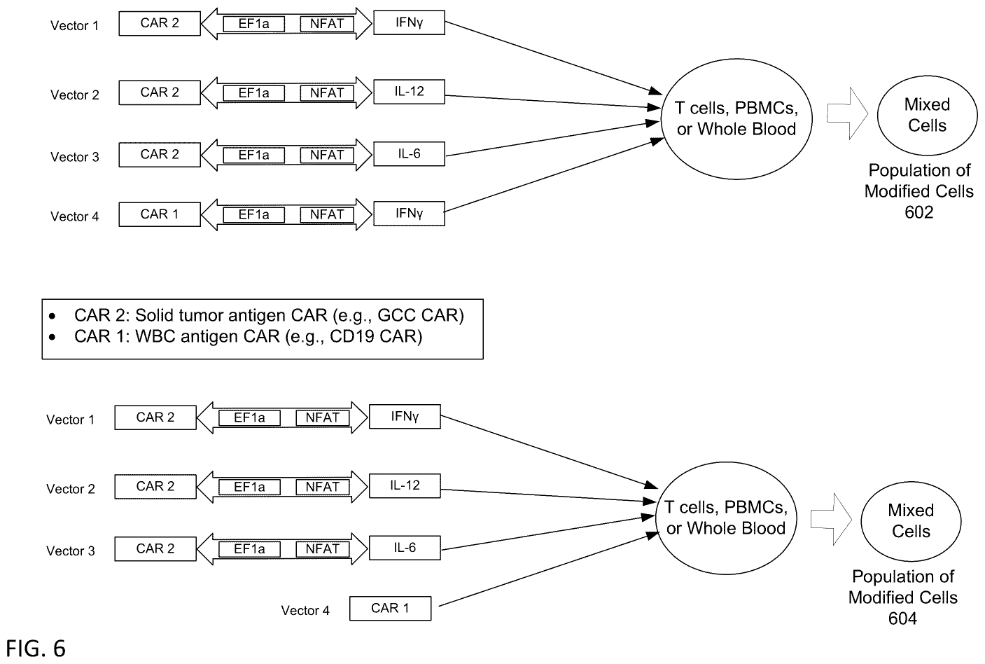

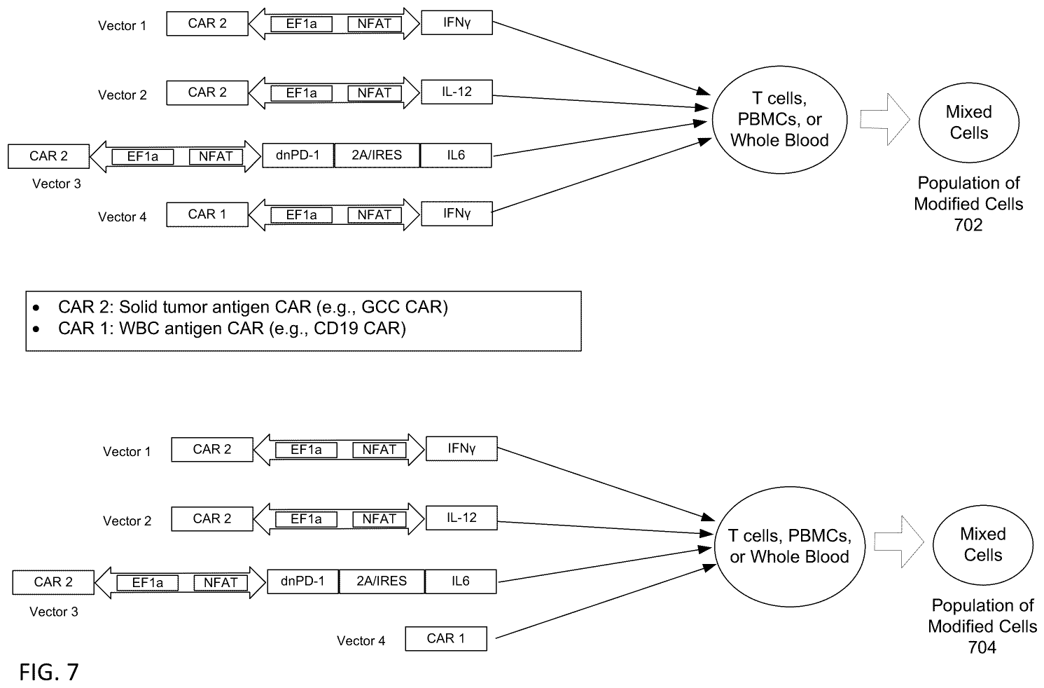

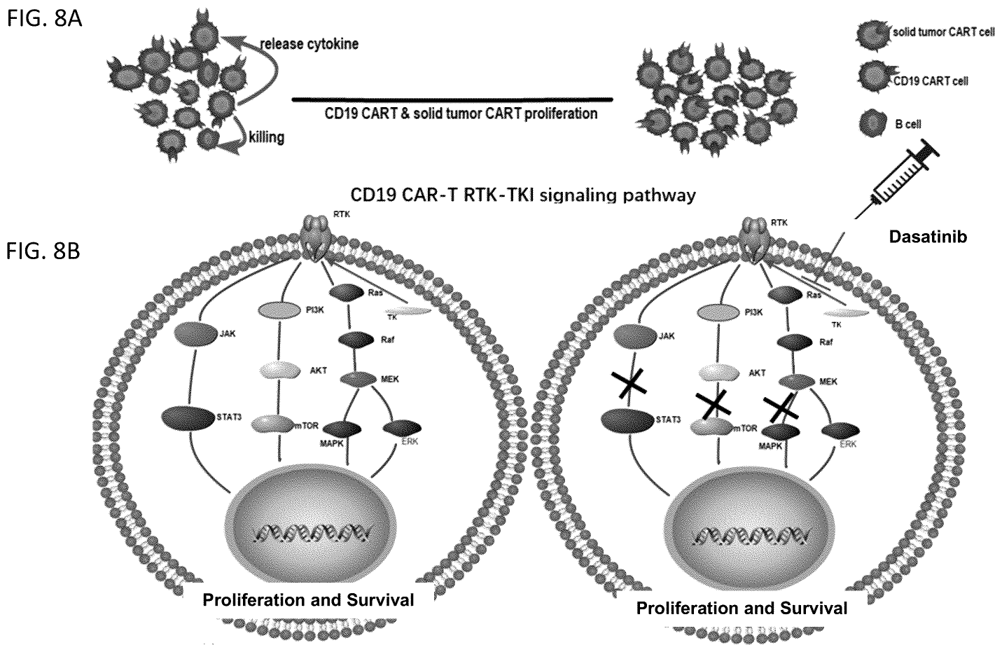

### DETAILED DESCRIPTION

Unless defined otherwise, all technical and scientific terms used herein have the same meaning as commonly understood by those of ordinary skill in the art to which the disclosure belongs. Although any method and material similar or equivalent to those described herein can be used in the practice or testing of the present disclosure, preferred methods and materials are described. For the purposes of the present disclosure, the following terms are defined below.

The articles “a” and “an” are used herein to refer to one or to more than one (i.e., to at least one) of the grammatical object of the article. By way of example, “an element” means one element or more than one element.

By “about” is meant a quantity, level, value, number, frequency, percentage, dimension, size, amount, weight or length that varies by as much as 20, 15, 10, 9, 8, 7, 6, 5, 4, 3, 2 or 1% to a reference quantity, level, value, number, frequency, percentage, dimension, size, amount, weight or length.

The term “activation,” as used herein, refers to the state of a cell that has been sufficiently stimulated to induce detectable cellular proliferation. Activation can also be associated with induced cytokine production and detectable effector functions. The term “activated T cells” refers to, among other things, T cells that are undergoing cell division.

The term “antibody” is used in the broadest sense and refers to monoclonal antibodies (including full length monoclonal antibodies), polyclonal antibodies, multi-specific antibodies (e.g., bispecific antibodies), and antibody fragments so long as they exhibit the desired biological activity or function. The antibodies in the present disclosure may exist in a variety of forms including, for example, polyclonal antibodies; monoclonal antibodies; Fv, Fab, Fab′, and F(ab′)2 fragments; as well as single chain antibodies and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, New York; Houston et al., 1988, Proc. Natl. Acad. Sci. USA 85:5879-5883; Bird et al., 1988, Science 242:423-426).

The term “antibody fragments” refers to a portion of a full-length antibody, for example, the antigen binding or variable region of the antibody. Other examples of antibody fragments include Fab, Fab′, F(ab′)2, and Fv fragments; diabodies; linear antibodies; single-chain antibody molecules; and multi-specific antibodies formed from antibody fragments.

The term “Fv” refers to the minimum antibody fragment containing a complete antigen-recognition and -binding site. This fragment consists of a dimer of one heavy- and one light-chain variable region domain in a tight, non-covalent association. From the folding of these two domains emanates six hypervariable loops (3 loops each from the H and L chain) that contribute amino acid residues for antigen binding and confer antigen binding specificity to the antibody. However, even a single variable domain (or half of an Fv including only three complementarity determining regions (CDRs) specific for an antigen) has the ability to recognize and bind antigen, although at a lower affinity than the entire binding site (the dimer).

An “antibody heavy chain,” as used herein, refers to the larger of the two types of polypeptide chains present in all antibody molecules in their naturally occurring conformations. An “antibody light chain,” as used herein, refers to the smaller of the two types of polypeptide chains present in all antibody molecules in their naturally occurring conformations. K and A light chains refer to the two major antibody light chain isotypes.

The term “synthetic antibody” refers to an antibody that is generated using recombinant DNA technology, such as, for example, an antibody expressed by a bacteriophage. The term also includes an antibody that has been generated by the synthesis of a DNA molecule encoding the antibody and the expression of the DNA molecule to obtain the antibody or to obtain an amino acid encoding the antibody. Synthetic DNA is obtained using available and well-known technology in the art.

The term “antigen” refers to a molecule that provokes an immune response, which may involve either antibody production, the activation of specific immunologically-competent cells, or both. Antigens include any macromolecule, including all proteins, peptides, or molecules derived from recombinant or genomic DNA. For example, DNA includes a nucleotide sequence or a partial nucleotide sequence encoding a protein or peptide that elicits an immune response, and therefore, encodes an “antigen” as the term is used herein. An antigen need not be encoded solely by a full-length nucleotide sequence of a gene. An antigen can be generated, synthesized, or derived from a biological sample including a tissue sample, a tumor sample, a cell, or a biological fluid.

The term “anti-tumor effect,” as used herein, refers to a biological effect associated with a decrease in tumor volume, a decrease in the number of tumor cells, a decrease in the number of metastases, decrease in tumor cell proliferation, decrease in tumor cell survival, an increase in life expectancy of a subject having tumor cells, or amelioration of various physiological symptoms associated with the cancerous condition. An “anti-tumor effect” can also be manifested by the ability of the peptides, polynucleotides, cells, and antibodies to prevent the occurrence of tumor in the first place.

The term “auto-antigen” refers to an endogenous antigen mistakenly recognized by the immune system as foreign. Auto-antigens include cellular proteins, phosphoproteins, cellular surface proteins, cellular lipids, nucleic acids, glycoproteins, including cell surface receptors.

The term “autologous” is used to describe a material derived from a subject that is subsequently re-introduced into the same subject.

The term “allogeneic” is used to describe a graft derived from a different subject of the same species. For example, a donor subject may be related or unrelated to the recipient subject, but the donor subject has immune system markers similar to the recipient subject.

The term “xenogeneic” is used to describe a graft derived from a subject of a different species. As an example, the donor subject is from a different species than a recipient subject, and the donor subject and the recipient subject can be genetically and immunologically incompatible.

The term “cancer” is used to refer to a disease characterized by the rapid and uncontrolled growth of aberrant cells. Cancer cells can spread locally or through the bloodstream and lymphatic system to other parts of the body. Examples of various cancers include breast cancer, prostate cancer, ovarian cancer, cervical cancer, skin cancer, pancreatic cancer, colorectal cancer, renal cancer, liver cancer, brain cancer, lymphoma, leukemia, lung cancer, and the like.

Throughout this specification, unless the context requires otherwise, the words “comprise,” “includes,” and “including” will be understood to imply the inclusion of a stated step or element or group of steps or elements but not the exclusion of any other step or element or group of steps or elements.

The phrase “consisting of” is meant to include, and is limited to, whatever follows the phrase “consisting of.” Thus, the phrase “consisting of” indicates that the listed elements are required or mandatory and that no other elements may be present.

The phrase “consisting essentially of” is meant to include any element listed after the phrase and can include other elements that do not interfere with or contribute to the activity or action specified in the disclosure for the listed elements. Thus, the phrase “consisting essentially of” indicates that the listed elements are required or mandatory, but those other elements are optional and may or may not be present depending upon whether they affect the listed elements' activity or action.

The terms “complementary” and “complementarity” refer to polynucleotides (i.e., a sequence of nucleotides) related by the base-pairing rules. For example, the sequence “A-G-T,” is complementary to the sequence “T-C-A.” Complementarity may be “partial,” in which only some of the nucleic acids' bases are matched according to the base pairing rules, or there may be “complete” or “total” complementarity between the nucleic acids. The degree of complementarity between nucleic acid strands has significant effects on the efficiency and strength of hybridization between nucleic acid strands.

The term “corresponds to” or “corresponding to” refers to (a) a polynucleotide having a nucleotide sequence that is substantially identical or complementary to all or a portion of a reference polynucleotide sequence or encoding an amino acid sequence identical to an amino acid sequence in a peptide or protein, or (b) a peptide or polypeptide having an amino acid sequence that is substantially identical to a sequence of amino acids in a reference peptide or protein.

The term “co-stimulatory ligand” refers to a molecule on an antigen-presenting cell (e.g., an APC, dendritic cell, B cell, and the like) that specifically binds a cognate co-stimulatory molecule on a T cell, thereby providing a signal which, in addition to the primary signal provided by, for instance, binding of a TCR/CD3 complex with an MHC molecule loaded with peptide, mediates a T cell response, including at least one of proliferation, activation, differentiation, and other cellular responses. A co-stimulatory ligand can include B7-1 (CD80), B7-2 (CD86), PD-L1, PD-L2, 4-1BBL, OX40L, inducible co-stimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, HVEM, a ligand for CD7, an agonist or antibody that binds the Toll ligand receptor, and a ligand that binds explicitly with B7-H3. A co-stimulatory ligand also includes, inter alia, an agonist or an antibody that specifically binds with a co-stimulatory molecule present on a T cell, such as CD27, CD28, 4-1BB, OX40, CD30, CD40, PD-1, ICOS, lymphocyte function-associated antigen-1 (LFA-1), CD2, CD7, LIGHT, NKG2C, B7-H3, and a ligand that specifically binds CD83.

The term “co-stimulatory molecule” refers to the cognate binding partner on a T cell that specifically binds with a co-stimulatory ligand, thereby mediating a co-stimulatory response by the T cell, such as proliferation. Co-stimulatory molecules include an MHC class I molecule, BTLA, and a Toll-like receptor.

The term “co-stimulatory signal” refers to a signal that, combined with a primary signal, such as TCR/CD3 ligation, leads to T cell proliferation and/or upregulation or downregulation of key molecules.

The terms “co-stimulatory signaling region”, “co-stimulatory domain”, and “co-stimulation domain” are used interchangeably to refer to one or more additional stimulatory domain in addition to a stimulatory or signaling domain such as CD3 zeta. The terms “stimulatory” or “signaling” domain (or region) are also used interchangeably, when referring, for example, to CD3 zeta, the primary signaling domain. In embodiments, the co-stimulatory signaling domain and the stimulatory signaling domain can be on the same molecule or different molecules in the same cell.

The terms “disease” and “condition” may be used interchangeably or may be different in that the particular malady or condition may not have a known causative agent (so that etiology has not yet been worked out), and it is therefore not yet recognized as a disease but only as an undesirable condition or syndrome, wherein a more or less specific set of symptoms have been identified by clinicians. The term “disease” is a state of health of a subject wherein the subject cannot maintain homeostasis and wherein if the disease is not ameliorated, the subject's health continues to deteriorate. In contrast, a “disorder” in a subject is a state of health in which the animal can maintain homeostasis, but in which the animal's state of health is less favorable than it would be in the absence of the disorder. Left untreated, a disorder does not necessarily cause a further decrease in the animal's state of health.

The term “effective” refers to adequate to accomplish a desired, expected, or intended result. For example, an “effective amount” in the context of treatment may be an amount of a compound sufficient to produce a therapeutic or prophylactic benefit.

The term “encoding” refers to the inherent property of specific sequences of nucleotides in a polynucleotide, such as a gene, a cDNA, or an mRNA, to serve as a template for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence (except that a “T” is replaced by a “U”) and is usually provided in sequence listings, and the non-coding strand, used as the template for transcription of a gene or cDNA, can be referred to as encoding the protein or other product of that gene or cDNA.

The term “exogenous” refers to a molecule that does not naturally occur in a wild-type cell or organism but is typically introduced into the cell by molecular biological techniques. Examples of exogenous polynucleotides include vectors, plasmids, and/or man-made nucleic acid constructs encoding the desired protein. With regard to polynucleotides and proteins, the term “endogenous” or “native” refers to naturally-occurring polynucleotide or amino acid sequences that may be found in a given wild-type cell or organism. Also, a particular polynucleotide sequence isolated from a first organism and transferred to a second organism by molecular biological techniques is typically considered an “exogenous” polynucleotide or amino acid sequence with respect to the second organism. In specific embodiments, polynucleotide sequences can be “introduced” by molecular biological techniques into a microorganism that already contains such a polynucleotide sequence, for instance, to create one or more additional copies of an otherwise naturally-occurring polynucleotide sequence, and thereby facilitate overexpression of the encoded polypeptide.

The term “expression or overexpression” refers to the transcription and/or translation of a particular nucleotide sequence into a precursor or mature protein, for example, driven by its promoter. “Overexpression” refers to the production of a gene product in transgenic organisms or cells that exceeds levels of production in normal or non-transformed organisms or cells. As defined herein, “expression” refers to expression or overexpression.

The term “expression vector” refers to a vector including a recombinant polynucleotide including expression control (regulatory) sequences operably linked to a nucleotide sequence to be expressed. An expression vector includes sufficient cis-acting elements for expression; other elements for expression can be supplied by the host cell or in an in vitro expression system. Expression vectors include all those known in the art, such as cosmids, plasmids (e.g., naked or contained in liposomes), and viruses (e.g., lentiviruses, retroviruses, adenoviruses, and adeno-associated viruses) that incorporate the recombinant polynucleotide.

Viruses can deliver nucleic acids into a cell in vitro and in vivo (in a subject). Examples of viruses useful for delivery of nucleic acids into cells include retrovirus, adenovirus, herpes simplex virus, vaccinia virus, and adeno-associated virus.

There also exist non-viral methods for delivering nucleic acids into a cell, for example, electroporation, gene gun, sonoporation, magnetofection, and the use of oligonucleotides, lipoplexes, dendrimers, and inorganic nanoparticles.

The term “homologous” refers to sequence similarity or sequence identity between two polypeptides or between two polynucleotides when a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared ×100. For example, if 6 of 10 of the positions in two sequences are matched or homologous, the two sequences are 60% homologous. By way of example, the DNA sequences ATTGCC and TATGGC share 50% homology. A comparison is made when two sequences are aligned to give maximum homology.

The term “immunoglobulin” or “Ig,” refers to a class of proteins that function as antibodies. The five members included in this class of proteins are IgA, IgG, IgM, IgD, and IgE. IgA is the primary antibody present in body secretions, such as saliva, tears, breast milk, gastrointestinal secretions, and mucus secretions of the respiratory and genitourinary tracts. IgG is the most common circulating antibody. IgM is the main immunoglobulin produced in the primary immune response in most subjects. It is the most efficient immunoglobulin in agglutination, complement fixation, and other antibody responses and is important in defense against bacteria and viruses. IgD is the immunoglobulin with no known antibody function but may serve as an antigen receptor. Finally, IgE is the immunoglobulin that mediates immediate hypersensitivity by causing the release of mediators from mast cells and basophils upon exposure to the allergen.

The term “isolated” refers to a material that is substantially or essentially free from components that normally accompany it in its native state. The material can be a cell or a macromolecule, such as a protein or nucleic acid. For example, an “isolated polynucleotide,” as used herein, refers to a polynucleotide, which has been purified from the sequences which flank it in a naturally-occurring state, e.g., a DNA fragment that has been removed from the sequences that are normally adjacent to the fragment. Alternatively, an “isolated peptide” or an “isolated polypeptide” and the like, as used herein, refer to in vitro isolation and/or purification of a peptide or polypeptide molecule from its natural cellular environment from association with other components of the cell.

The term “substantially purified” refers to a material that is substantially free from components normally associated with it in its native state. For example, a substantially purified cell refers to a cell that has been separated from other cell types with which it is normally associated in its naturally occurring or native state. In some instances, a population of substantially purified cells refers to a homogenous population of cells. In other instances, this term refers simply to a cell that has been separated from the cells with which they are naturally associated in their natural state. In embodiments, the cells are cultured in vitro. In embodiments, the cells are not cultured in vitro.

In the context of the present disclosure, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and encode the same amino acid sequence. The phrase nucleotide sequence that encodes a protein or an RNA may also include introns to the extent that the nucleotide sequence encoding the protein may contain an intron(s) in some version.

The term “lentivirus” refers to a genus of the Retroviridae family. Lentiviruses are unique among the retroviruses in infecting non-dividing cells; they can deliver a significant amount of genetic information into the DNA of the host cell, so they are one of the most efficient methods of a gene delivery vector. Moreover, lentiviruses enable the integration of genetic information into the host chromosome, resulting in stably transduced genetic information. HIV, SIV, and FIV are all examples of lentiviruses. Vectors derived from lentiviruses offer the means to achieve significant levels of gene transfer in vivo.

The term “modulating” refers to mediating a detectable increase or decrease in the level of a response in a subject compared with the level of a response in the subject in the absence of a treatment or compound and/or compared with the level of a response in an otherwise identical but untreated subject. The term encompasses perturbing and/or affecting a native signal or response, thereby mediating a beneficial therapeutic response in a subject, preferably a human.

Nucleic acid is “operably linked” when placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence, or a ribosome binding site is operably linked to a coding sequence if it is positioned to facilitate translation.

The term “under transcriptional control” refers to a promoter being operably linked to and in the correct location and orientation in relation to a polynucleotide to control (regulate) the initiation of transcription by RNA polymerase and expression of the polynucleotide.

The term “overexpressed” tumor antigen or “overexpression” of the tumor antigen is intended to indicate an abnormal level of expression of the tumor antigen in a cell from a disease area such as a solid tumor within a specific tissue or organ of the patient relative to the level of expression in a normal cell from that tissue or organ. Patients having a solid tumor or a hematological malignancy characterized by overexpression of the tumor antigen can be determined by standard assays known in the art.

Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas). Examples of solid tumors, such as sarcomas and carcinomas, include fibrosarcoma, myxosarcoma, liposarcoma, chondrosarcoma, osteosarcoma, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme), astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma, and brain metastases).

A solid tumor antigen is an antigen expressed on a solid tumor. In embodiments, solid tumor antigens are also expressed at low levels on healthy tissue. Examples of solid tumor antigens and their related disease tumors are provided in Table 1.

TABLE 1

| Solid Tumor antigen | Disease tumor |
|---|---|
| PRLR | Breast Cancer |
| CLCA1 | colorectal Cancer |
| MUC12 | colorectal Cancer |
| GUCY2C | colorectal Cancer |
| GPR35 | colorectal Cancer |
| CR1L | Gastric Cancer |
| MUC 17 | Gastric Cancer |
| TMPRSS11B | esophageal Cancer |
| MUC21 | esophageal Cancer |
| TMPRSS11E | esophageal Cancer |
| CD207 | bladder Cancer |
| SLC30A8 | pancreatic Cancer |
| CFC1 | pancreatic Cancer |
| SLC12A3 | Cervical Cancer |
| SSTR1 | Cervical tumor |
| GPR27 | Ovary tumor |
| FZD10 | Ovary tumor |
| TSHR | Thyroid Tumor |
| SIGLEC15 | Urothelial cancer |
| SLC6A3 | Renal cancer |
| KISS1R | Renal cancer |
| QRFPR | Renal cancer: |
| GPR119 | Pancreatic cancer |
| CLDN6 | Endometrial cancer/Urothelial cancer |
| UPK2 | Urothelial cancer (including bladder cancer) |
| ADAM12 | Breast cancer, pancreatic cancer, and the like |
| SLC45A3 | Prostate cancer |
| ACPP | Prostate cancer |
| MUC21 | Esophageal cancer |
| MUC 16 | Ovarian cancer |
| MS4A12 | Colorectal cancer |
| ALPP | Endometrial cancer |
| CEA | Colorectal carcinoma |
| EphA2 | Glioma |
| FAP | Mesotelioma |
| GPC3 | Lung squamous cell carcinoma |
| IL 13-Rα2 | Glioma |
| Mesothelin | Metastatic cancer |
| PSMA | Prostate cancer |
| ROR1 | Breast lung carcinoma |
| VEGFR-II | Metastatic cancer |
| GD2 | Neuroblastoma |
| FR-α | Ovarian carcinoma |
| ErbB2 | Carcinomasb |
| EpCAM | Carcinomasa |
| EGFRvIII | Glioma-Glioblastoma |
| EGFR | Glioma-NSCL cancer |
| tMUC1 | Cholangiocarcinoma, Pancreatic cancer, |
| | Breast |
| PSCA | pancreas, stomach, or prostate cancer |
| FCER2, GPR18, FCRLA, | breast cancer |
| CXCR5, FCRL3, FCRL2, | |
| HTR3A, and CLEC17A | |
| TRPMI, SLC45A2, and | lymphoma |
| SLC24A5 | |
| DPEP3 | melanoma |
| KCNK16 | ovarian, testis |
| LIM2 or KCNV2 | pancreatic |
| SLC26A4 | thyroid cancer |
| CD171 | Neuroblastoma |
| Glypican-3 | Sarcoma |
| IL-13 | Glioma |
| CD79a/b | Lymphoma |
| MAGE A4 | Lung cancer |

The term “parenteral administration” of a composition includes, e.g., subcutaneous (s.c.), intravenous (i.v.), intramuscular (i.m.), intrasternal injection, or infusion techniques.

The terms “patient,” “subject,” “individual,” and the like are used interchangeably herein and refer to any human or animal, amenable to the methods described herein. In certain non-limiting embodiments, the patient, subject, or individual is a human or animal. For example, in embodiments, the term “subject” includes living organisms in which an immune response can be elicited (e.g., mammals). Examples of subjects include humans, and animals, such as dogs, cats, mice, rats, and transgenic species.

A subject in need of treatment or need thereof includes a subject having a disease, condition, or disorder that needs to be treated. A subject in need also includes a subject that needs treatment to prevent disease, condition, or disorder.

“Polynucleotide” or “nucleic acid” refers to mRNA, RNA, cRNA, rRNA, cDNA, or DNA. The term typically refers to a polymeric form of nucleotides of at least 10 bases in length, either ribonucleotides or deoxynucleotides, or a modified form of either type of nucleotide. The term includes all forms of nucleic acids, including single and double-stranded forms of nucleic acids.

The terms “polynucleotide variant” and “variant,” and the like refer to polynucleotides displaying substantial sequence identity with a reference polynucleotide sequence or polynucleotides that hybridize with a reference sequence under stringent conditions that are defined hereinafter. These terms also encompass polynucleotides distinguished from a reference polynucleotide by adding, deleting, or substituting at least one nucleotide. Accordingly, the terms “polynucleotide variant” and “variant” include polynucleotides in which one or more nucleotides have been added, deleted, or replaced with different nucleotides. In this regard, it is well understood in the art that certain alterations inclusive of mutations, additions, deletions, and substitutions can be made to a reference polynucleotide whereby the altered polynucleotide retains the biological function or activity of the reference polynucleotide or has increased activity in relation to the reference polynucleotide (i.e., optimized). Polynucleotide variants include, for example, polynucleotides having at least 50% (and at least 51% to at least 99% and all integer percentages in between, e.g., 90%, 95%, or 98%) sequence identity with a reference polynucleotide sequence described herein. The terms “polynucleotide variant” and “variant” also include naturally-occurring allelic variants and orthologs.

The terms “polypeptide,” “polypeptide fragment,” “peptide,” and “protein” are used interchangeably herein to refer to a polymer of amino acid residues and variants and synthetic analogues of the same. Thus, these terms apply to amino acid polymers in which one or more amino acid residues are synthetic non-naturally occurring amino acids, such as a chemical analogue of a corresponding naturally occurring amino acid, as well as to naturally-occurring amino acid polymers. In certain aspects, polypeptides may include enzymatic polypeptides, or “enzymes,” which typically catalyze (i.e., increase the rate of) various chemical reactions.

The term “polypeptide variant” refers to polypeptides that are distinguished from a reference polypeptide sequence by the addition, deletion, or substitution of at least one amino acid residue. In certain embodiments, a polypeptide variant is distinguished from a reference polypeptide by one or more substitutions, which may be conservative or non-conservative. In certain embodiments, the polypeptide variant comprises conservative substitutions. In this regard, it is well understood in the art that some amino acids may be changed to others with broadly similar properties without changing the nature of the polypeptide activity. Polypeptide variants also encompass polypeptides in which one or more amino acids have been added, deleted, or replaced with different amino acid residues.

The term “promoter” refers to a DNA sequence recognized by the cell's synthetic machinery or introduced synthetic machinery required to initiate the specific transcription of a polynucleotide sequence. The term “expression control (regulatory) sequences” refers to DNA sequences necessary to express an operably linked coding sequence in a particular host organism. The control sequences that are suitable for prokaryotes, for example, include a promoter, optionally an operator sequence, and a ribosome binding site. In addition, eukaryotic cells utilize promoters, polyadenylation signals, and enhancers.

The term “bind,” “binds,” or “interacts with” refers to a molecule recognizing and adhering to a second molecule in a sample or organism but does not substantially recognize or adhere to other structurally unrelated molecules in the sample. The term “specifically binds,” as used herein with respect to an antibody, refers to an antibody that recognizes a specific antigen but does not substantially recognize or bind other molecules in a sample. For example, an antibody that specifically binds an antigen from one species may also bind that antigen from one or more species. But, such cross-species reactivity does not alter an antibody's classification as specific. In another example, an antibody that specifically binds an antigen may also bind different allelic forms of the antigen. However, such cross reactivity does not alter the classification of an antibody as specific. In some instances, the terms “specific binding” or “specifically binding” can be used about the interaction of an antibody, a protein, or a peptide with a second chemical species, to mean that the interaction is dependent upon the presence of a particular structure (e.g., an antigenic determinant or epitope) on the chemical species; for example, an antibody recognizes and binds a specific protein structure rather than to any protein. If an antibody is specific for epitope “A,” the presence of a molecule containing epitope A (or free, unlabeled A), in a reaction containing labeled “A” and the antibody, will reduce the amount of labeled A bound to the antibody.

By “statistically significant,” it is meant that the result was unlikely to have occurred by chance. Statistical significance can be determined by any method known in art. Commonly used significance measures include the p-value, which is the frequency or probability with which the observed event would occur if the null hypothesis were true. The null hypothesis is rejected if the obtained p-value is smaller than the significance level. In simple cases, the significance level is defined at a p-value of 0.05 or less. A “decreased” or “reduced” or “lesser” amount is typically a “statistically significant” or a physiologically significant amount. It may include a decrease that is about 1.1, 1.2, 1.3, 1.4, 1.5, 1.6 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 1.5, 1.6, 1.7. 1.8, etc.) an amount or level described herein.

The term “stimulation” refers to a primary response induced by binding a stimulatory molecule (e.g., a TCR/CD3 complex) with its cognate ligand, thereby mediating a signal transduction event, such as signal transduction via the TCR/CD3 complex. In addition, stimulation can mediate altered expression of certain molecules, such as downregulation of TGF-β and/or reorganization of cytoskeletal structures.

The term “stimulatory molecule” refers to a molecule on a T cell that specifically binds a cognate stimulatory ligand present on an antigen presenting cell. For example, a functional signaling domain derived from a stimulatory molecule is the zeta chain associated with the T cell receptor complex. The stimulatory molecule includes a domain responsible for signal transduction.

The term “stimulatory ligand” refers to a ligand that when present on an antigen-presenting cell (e.g., an APC, a dendritic cell, a B-cell, and the like.) can specifically bind with a cognate binding partner (referred to herein as a “stimulatory molecule”) on a cell, for example, a T cell, thereby mediating a primary response by the T cell, including activation, initiation of an immune response, proliferation, and similar processes. Stimulatory ligands are well-known in the art and encompass, inter alia, an MHC Class I molecule loaded with a peptide, an anti-CD3 antibody, a superagonist anti-CD28 antibody, and a superagonist anti-CD2 antibody.

The term “therapeutic” refers to treatment and/or prophylaxis. A therapeutic effect is obtained by suppression, remission, or eradication of a disease state or alleviating the symptoms of a disease state.

The term “therapeutically effective amount” refers to the amount of the subject compound that will elicit the biological or medical response of a tissue, system, or subject sought by the researcher, veterinarian, medical doctor or another clinician. The term “therapeutically effective amount” includes that amount of a compound that, when administered, is sufficient to prevent the development of, or alleviate to some extent, one or more of the signs or symptoms of the disorder or disease being treated. The therapeutically effective amount will vary depending on the compound, disease, severity, age, weight, etc., of the subject to be treated.

The term “treat a disease” refers to reducing the frequency or severity of at least one sign or symptom of a disease or disorder experienced by a subject.

The term “transfected” or “transformed” or “transduced” refers to a process by which an exogenous nucleic acid is transferred or introduced into the host cell. A “transfected” or “transformed” or “transduced” cell has been transfected, transformed, or transduced with the exogenous nucleic acid. The cell includes the primary subject cell and its progeny.

The term “vector” refers to a polynucleotide that comprises an isolated nucleic acid and can be used to deliver the isolated nucleic acid to the interior of a cell. Numerous vectors are known in the art including linear polynucleotides, polynucleotides associated with ionic or amphiphilic compounds, plasmids, and viruses. Thus, the term “vector” includes an autonomously replicating plasmid or a virus. The term also includes non-plasmid and non-viral compounds that facilitate the transfer of nucleic acid into cells, such as polylysine compounds, liposomes, and the like. Examples of viral vectors include adenoviral vectors, adeno-associated virus vectors, retroviral vectors, and others. For example, lentiviruses are complex retroviruses, which, in addition to the common retroviral genes gag, pol, and env, contain other genes with regulatory or structural functions. Lentiviral vectors are well known in the art. Some examples of lentivirus include the Human Immunodeficiency Viruses: HIV-1, HIV-2, and the Simian Immunodeficiency Virus: SIV. Lentiviral vectors have been generated by multiply attenuating the HIV virulence genes, for example, the genes env, vif, vpr, vpu, and nef are deleted, making the vector biologically safe.

Ranges: throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges and individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, 1, 2, 2.7, 3, 4, 5, 5.3, and 6. This applies regardless of the breadth of the range.

A “chimeric antigen receptor” (CAR) molecule is a recombinant polypeptide that includes at least an extracellular domain, a transmembrane domain, and a cytoplasmic or intracellular domain. In embodiments, the domains of the CAR are on the same polypeptide chain, for example, a chimeric fusion protein. However, in embodiments, the domains are on different polypeptide chains, for example, the domains are not contiguous.

The extracellular domain of a CAR molecule includes an antigen binding domain. The antigen binding domain is for expanding and/or maintaining the modified cells, such as CAR T cells, or killing a tumor cell, such as a solid tumor. In embodiments, the antigen binding domain for expanding and/or maintaining modified cells binds an antigen, for example, a cell surface molecule or marker, on the surface of a WBC. In embodiments, the WBC is at least one of GMP (granulocyte macrophage precursor), MDP (monocyte-macrophage/dendritic cell precursors), cMoP (common monocyte precursor), basophil, eosinophil, neutrophil, SatM (Segerate-nucleus-containing atypical monocyte), macrophage, monocyte, CDP (common dendritic cell precursor), cDC (conventional DC), pDC (plasmacytoid DC), CLP (common lymphocyte precursor), B cell, ILC (Innate Lymphocyte), NK cell, megakaryocyte, myeloblast, pro-myelocyte, myelocyte, meta-myelocyte, band cells, lymphoblast, prolymphocyte, monoblast, megakaryoblast, promegakaryocyte, megakaryocyte, platelets, or MSDC (Myeloid-derived suppressor cell). In embodiments, the WBC is a granulocyte, monocyte, and or lymphocyte. In embodiments, the WBC is a lymphocyte, for example, a B cell. In embodiments, the WBC is a B cell. In embodiments, the cell surface molecule of a B cell includes CD19, CD22, CD20, BCMA, CD5, CD7, CD2, CD16, CD56, CD30, CD14, CD68, CD11 b, CD18, CD169, CD1c, CD33, CD38, CD138, or CD13. In embodiments, the cell surface molecule of the B cell is CD19, CD20, CD22, or BCMA. In embodiments, the cell surface molecule of the B cell is CD19.

The cells described herein, including modified cells such as CAR and T cells, can be derived from stem cells. Stem cells may be adult stem cells, embryonic stem cells, more particularly non-human stem cells, cord blood stem cells, progenitor cells, bone marrow stem cells, induced pluripotent stem cells, totipotent stem cells, or hematopoietic stem cells. A modified cell may also be a dendritic cell, an NK-cell, a B-cell, or a T cell selected from the group consisting of inflammatory T-lymphocytes, cytotoxic T-lymphocytes, regulatory T lymphocytes, or helper T-lymphocytes. In embodiments, Modified cells may be derived from the group consisting of CD4+ T lymphocytes and CD8+ T lymphocytes. Prior to expansion and genetic modification of the cells of the invention, a source of cells may be obtained from a subject through various non-limiting methods. T cells may be obtained from a number of non-limiting sources, including peripheral blood mononuclear cells, bone marrow, lymph node tissue, cord blood, thymus tissue, tissue from a site of infection, ascites, pleural effusion, spleen tissue, and tumors. In certain embodiments of the present invention, any T cell lines available and known to those skilled in the art may be used. In embodiments, modified cells may be derived from a healthy donor, from a patient diagnosed with cancer, or from a patient diagnosed with an infection. In embodiments, a modified cell is part of a mixed population of cells that present different phenotypic characteristics.

A population of cells refers to a group of two or more cells. The cells of the population could be the same, such that the population is a homogenous population of cells. The cells of the population could be different, such that the population is a mixed population or a heterogeneous population of cells. For example, a mixed population of cells could include modified cells comprising a first CAR and cells comprising a second CAR, wherein the first CAR and the second CAR bind different antigens.

The term “stem cell” refers to certain types of cells that have the capacity for self-renewal and the ability to differentiate into other kinds (s) of a cell. For example, a stem cell gives rise either to two daughter stem cells (as occurs in vitro with embryonic stem cells in culture) or to one stem cell and a cell that undergoes differentiation (as occurs, e.g., in hematopoietic stem cells, which give rise to blood cells). Different categories of stem cells may be distinguished based on their origin and/or on the extent of their capacity for differentiation into other types of cells. For example, stem cells may include embryonic stem (ES) cells (i.e., pluripotent stem cells), somatic stem cells, induced pluripotent stem cells, and other types of stem cells.

The pluripotent embryonic stem cells are found in the inner cell mass of a blastocyst and have an innate capacity for differentiation. For example, pluripotent embryonic stem cells can form any type of cell in the body. When grown in vitro for long periods, ES cells maintain pluripotency as progeny cells retain the potential for multilineage differentiation.

Somatic stem cells can include fetal stem cells (from the fetus) and adult stem cells (found in various tissues, such as bone marrow). These cells have been regarded as having a capacity for differentiation lower than that of the pluripotent ES cells—with the capacity of fetal stem cells being greater than that of adult stem cells. Somatic stem cells apparently differentiate into only a limited number of types of cells and have been described as multipotent. The “tissue-specific” stem cells normally give rise to only one cell type. For example, embryonic stem cells may be differentiated into blood stem cells (e.g., Hematopoietic stem cells (HSCs)), which may be further differentiated into various blood cells (e.g., red blood cells, platelets, white blood cells, etc.).

Induced pluripotent stem cells (i.e., iPS cells or iPSCs) may include a type of pluripotent stem cell artificially derived from a non-pluripotent cell (e.g., an adult somatic cell) by inducing an expression of specific genes. Induced pluripotent stem cells are similar to natural pluripotent stem cells, such as embryonic stem (ES) cells, in many aspects, such as the expression of certain stem cell genes and proteins, chromatin methylation patterns, doubling time, embryoid body formation, teratoma formation, viable chimera formation, and potency and differentiability. Induced pluripotent cells can be obtained from adult stomach, liver, skin, and blood cells.

In embodiments, the antigen binding domain for killing a tumor binds an antigen on the surface of a tumor, for example, a tumor antigen or tumor marker. Tumor antigens are proteins produced by tumor cells that elicit an immune response, particularly T cell mediated immune responses. Tumor antigens are well known in the art and include, for example, tumor associated MUC1 (tMUC1), a glioma-associated antigen, carcinoembryonic antigen (CEA), β-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, Her2/neu, surviving, telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor, CD19, and mesothelin. For example, when the tumor antigen is CD19, the CAR thereof can be referred to as CD19 CAR or 19CAR, a CAR molecule that includes an antigen binding domain that binds CD19.

In embodiments, the extracellular antigen binding domain of a CAR includes at least one scFv or at least a single domain antibody. As an example, there can be two scFvs on a CAR. The scFv includes a light chain variable (VL) region and a heavy chain variable (VH) region of a target antigen-specific monoclonal antibody joined by a flexible linker. Single chain variable region fragments can be made by linking light and/or heavy chain variable regions by using a short linking peptide (Bird et al., Science 242:423-426, 1988). An example of a linking peptide is the GS linker, which bridges approximately 3.5 nm between the carboxy terminus of one variable region and the amino terminus of the other variable region. Linkers of other sequences have been designed and used (Bird et al., 1988, supra). In general, linkers can be short, flexible polypeptides and preferably comprise about 20 or fewer amino acid residues. The single chain variants can be produced either recombinantly or synthetically. For synthetic production of scFv, an automated synthesizer can be used. For recombinant production of scFv, a suitable plasmid containing a polynucleotide that encodes the scFv can be introduced into a suitable host cell, either eukaryotic, such as yeast, plant, insect, or mammalian cells, or prokaryotic, such as E. coli. Polynucleotides encoding the scFv of interest can be made by routine manipulations such as ligation of polynucleotides. The resultant scFv can be isolated using standard protein purification techniques known in the art.

The cytoplasmic domain of the CAR molecules described herein includes one or more co-stimulatory domains and one or more signaling domains. The one or more signaling domains including the co-stimulatory and primary signaling domains transmit the signal and activate molecules, such as T cells, in response to antigen binding. The one or more co-stimulatory domains are derived from stimulatory molecules and/or co-stimulatory molecules, and the signaling domain is derived from a primary signaling domain, such as the CD3 zeta domain. In embodiments, the signaling domain further includes one or more functional signaling domains derived from a co-stimulatory molecule. In embodiments, the co-stimulatory molecules are cell surface molecules (other than antigens receptors or their ligands) required to activate a cellular response to an antigen.

In embodiments, the co-stimulatory domain includes the intracellular domain of CD27, CD28, 4-1BB, OX40, CD30, CD40, PD-1, ICOS, lymphocyte function-associated antigen-1 (LFA-1), CD2, CD7, LIGHT, NKG2C, B7-H3, a ligand that specifically binds with CD83, or any combination thereof. The signaling domain includes a CD3 zeta domain derived from a T cell receptor in embodiments.

The CAR molecules described herein also include a transmembrane domain. The incorporation of a transmembrane domain in the CAR molecules stabilizes the molecule. In embodiments, the transmembrane domain of the CAR molecules is the transmembrane domain of a CD28 or 4-1BB molecule.

Between the extracellular domain and the transmembrane domain of the CAR, there may be incorporated a spacer domain. As used herein, the term “spacer domain” generally means any oligo- or polypeptide that functions to link the transmembrane domain to the extracellular domain and/or the cytoplasmic domain on the polypeptide chain. A spacer domain may include up to 300 amino acids, preferably 10 to 100 amino acids, and most preferably 25 to 50 amino acids.

The present disclosure further describes methods or compositions for treating cancer using cells derived from tumor-infiltrating lymphocytes (TILs). In embodiments, a T cell clone that expresses a TCR with a high affinity for the target antigen may be isolated. For example, TILs or peripheral blood mononuclear cells (PBMCs) can be cultured in the presence of antigen-presenting cells (APCs) pulsed with a peptide representing an epitope known to elicit a dominant T cell response when presented in the context of a defined HLA allele. High-affinity clones may be selected based on MHC-peptide tetramer staining and/or the ability to recognize and lyse target cells pulsed with low titrated concentrations of cognate peptide antigen. After the clone has been selected, the TCRα and TCRβ chains or TCRγ and TCRδ chains are identified and isolated by molecular cloning. For example, for TCRα and TCRβ chains, the TCRα and TCR gene sequences are then used to generate an expression construct that ideally promotes stable, high-level expression of both TCRβ chains in human T cells. For example, the transduction vehicle, a gammaretrovirus or lentivirus, can then be generated and tested for functionality (antigen specificity and functional avidity) and used to produce a clinical lot of the vector. An aliquot of the final product can then be used to transduce the target T cell population (generally purified from patient PBMCs), which is expanded before infusion into the patient.

Various methods may be implemented to obtain genes encoding tumor-reactive TCR. More information is provided in Kershaw et al., Clin Transl Immunology. 2014 May; 3(5): e16. In embodiments, specific TCR can be derived from spontaneously occurring tumor-specific T cells in patients. Antigens included in this category include the melanocyte differentiation antigens MART-1 and gp100 and the MAGE antigens and NY-ESO-1, with expression in a broader range of cancers. In addition, TCRs specific for viral-associated malignancies can also be isolated, as long as viral proteins are expressed by transformed cells. Malignancies in this category include liver and cervical cancer, hepatitis and papilloma viruses, and Epstein-Barr virus-associated malignancies. In embodiments, target antigens of the TCR include CEA (e.g., for colorectal cancer), gp100, MART-1, p53 (e.g., for melanoma), MAGE-A3 (e.g., melanoma, esophageal and synovial sarcoma), and NY-ESO-1 (e.g., for melanoma and sarcoma as well as multiple myelomas).

In embodiments, preparation and transfusion of tumor infiltrating lymphocytes (TIL) may be implemented in the following manner. For example, tumor tissue from surgical or biopsy specimens can be obtained under aseptic conditions and transported to the cell culture chamber in an ice box. Necrotic tissue and adipose tissue can be removed. The tumor tissue can be cut into small pieces of about 1-3 cubic millimeters. Collagenase, hyaluronidase, and DNA enzyme can be added and digested overnight at 4° C. Filtering with a 0.2 μm filter, cells can be separated and collected by lymphocyte separation fluid under 1500 rpm for 5 min. Expanding the cells in a culture medium comprising PHA, 2-mercaptoethanol, and CD3 monoclonal antibody, and a small dose of IL-2 (10-20 IU/ml) may be added to induce activation and proliferation. The cell density may be carefully measured and maintained within the range of 0.5-2×10⁶/ml for 7-14 days at a temperature of 37° C. with 5% CO₂.

TIL positive cells can kill homologous cancer cells can be screened out by co-culture. The TIL-positive cells can be amplified in a serum-free medium containing a high dose of IL-2 (5000-6000 IU/ml) until greater than 1×10¹¹ TILs can be obtained. To administer TILs, they are first collected in saline using continuous-flow centrifugation and then filtered through a platelet-administration set into a 200-300 mL volume containing 5% albumin and 450000 IU of IL-2. The TILs can be infused into patients through a central venous catheter over a period of 30-60 minutes. In embodiments, TILs can be infused in two to four separate bags, and the individual infusions can be separated by several hours.

The present disclosure further describes a method of enhancing T cell response caused by CAR T/TILs/TCR based therapies using delivery of the antigen corresponding to these therapies. For example, GUCY2C or, at least, the extracellular domain of GUCY2C may be delivered to patients' bodies to enhance GUCY2C CAR T cells' anti-tumor activities, an increase in T cell response. In embodiments, the increase in T cell response is based on the number of copies of CAR(s) and/or the amount of cytokine released (e.g., IL-6 and IFN-γ. In embodiments, the T cell response comprises cytokine releases, cell expansion, and/or activation levels. In embodiments, the first vector further comprises a polynucleotide encoding IL-6 or IFNγ, or a combination thereof. In embodiments, the first vector further comprises a polynucleotide encoding IL-12. In embodiments, the polynucleotide comprises a polynucleotide encoding NFAT and/or VHL. In embodiments, the population of modified cells comprises cells expressing the first binding molecule and IL-6 or IFNγ, or a combination thereof, cells expressing the second binding molecules, cells expressing the first and second molecules, and/or cells expressing the first binding molecule and IL-12. In embodiments, the population of modified cells comprises cells expressing the second binding molecule and IL-6 or IFNγ, or a combination thereof, cells expressing the second binding molecules, cells expressing the first and second molecules, and/or cells expressing the first binding molecule and IL-12. In embodiments, the population of modified cells comprises cells expressing the second binding molecule and IL-6 or IFNγ, or a combination thereof, cells expressing the second binding molecules, cells expressing the first and second molecules, and/or cells expressing the second binding molecule and IL-12. In embodiments, the population of modified cells comprises cells expressing a dominant negative form of PD-1. The antigen may be formulated as a form of a vaccine. Examples of vaccines include DCs, including the antigen, amph-ligand, and a nanoparticle RNA vaccine. More information about the vaccine examples may be found at E Snook, A. “Companion vaccines for CAR T-cell therapy: applying basic immunology to enhance therapeutic efficacy,” Future Medicinal Chemistry, V. 12, No. 15, 2020, pp. 1359-62, which is incorporated by its entirety.

In embodiments, the CAR molecules' cytoplasmic domain described herein comprises a co-stimulatory domain and a CD3 zeta domain. In embodiments, the CAR molecules described herein may include a co-stimulatory domain without a corresponding component of the CD3 zeta domain. In embodiments, the CAR molecules described herein may include a CD3 zeta domain without a co-stimulatory domain.

In embodiments, the modified cell comprises a dominant negative variant of a receptor of programmed death 1 (PD-1), cytotoxic T lymphocyte antigen-4 (CTLA-4), B- and T-lymphocyte attenuator (BTLA), T cell immunoglobulin mucin-3 (TIM-3), lymphocyte-activation protein 3 (LAG-3), T cell immunoreceptor with Ig and ITIM domains (TIGIT), leukocyte-associated immunoglobulin-like receptor 1 (LAIRI), natural killer cell receptor 2B4 (2B4), or CD 160. In embodiments, the modified cell further comprises a nucleic acid sequence encoding a suicide gene, and/or the suicide gene comprises an HSV-TK suicide gene system. In embodiments, the isolated T cell comprises a reduced amount of TCR compared to the corresponding wide-type T cell.

Dominant negative mutations have an altered gene product that antagonizes the wild-type allele. These mutations usually result in an altered molecular function (often inactive) and are characterized by a dominant or semi-dominant phenotype. In embodiments, the modified cells described herein comprise the dominant negative (DN) form of the PD-1 receptor. In embodiments, the expression of the DN PD-1 receptor in the modified cells described herein is regulated by an inducible gene expression system. In embodiments, the inducible gene expression system is a lac system, a tetracycline system, or a galactose system.

The present disclosure describes pharmaceutical compositions. The pharmaceutical compositions include one or more of the following: CAR molecules, TCR molecules, modified CAR T cells, modified cells comprising CAR or TCR, mix population of modified cells, nucleic acids, and vectors described herein. Pharmaceutical compositions are administered appropriately to the disease to be treated (or prevented). Such factors will determine the quantity and frequency of administration as the patient's condition and the type and severity of the patient's disease, although clinical trials may determine appropriate dosages.

The term “pharmaceutically acceptable” means approved by a regulatory agency of the U.S. Federal or a state government or the EMA (European Medicines Agency) or listed in the U.S. Pharmacopeia Pharmacopeia (United States Pharmacopeia-33/National Formulary-28 Reissue, published by the United States Pharmacopeia Convention, Inc., Rockville Md., publication date: April 2010) or other generally recognized pharmacopeia for use in animals, and more particularly in humans.

The term “carrier” refers to a diluent, adjuvant {e.g., Freund's adjuvant (complete and incomplete)), excipient, or vehicle with which the therapeutic is administered. Pharmaceutical carriers can be sterile liquids, such as water and oils, including petroleum, animal, vegetable, or synthetic origins, such as peanut oil, soybean oil, mineral oil, sesame oil, and the like. Water is a preferred carrier when the pharmaceutical composition is administered intravenously. Saline solutions and aqueous dextrose and glycerol can also be used as liquid carriers, particularly for injectable solutions.

The present disclosure also describes a pharmaceutical composition comprising the first and the second population of cells described herein. The pharmaceutical composition described herein, comprising a first population of cells comprising a first antigen binding molecule and a second population of cells comprising a second antigen binding domain, are suitable for cancer therapy. For example, the binding of a first antigen binding molecule with an antigen enhances the expansion of the cells suitable for cancer therapy.

The present disclosure also describes a method for enhancing cancer therapy using the cells described herein suitable for cancer therapy. The method comprises administering an effective amount of a first composition to the subject having a form of cancer expressing a tumor antigen, the first composition comprising a population of cells (e.g., T cells) comprising an antigen binding molecule (e.g., CAR) binding an antigen; and administering an effective amount of a second composition to the subject, the second composition comprising the antigen in the form of vaccines (e.g., DC-antigen and nanoparticle-mRNA). The first and second compositions can be performed simultaneously or separately, for example, sequentially. More information about the cells suitable for cancer therapy can be found at Eyileten, C., Majchrzak, K., Pilch, Z., et al. “Immune Cells in Cancer Therapy and Drug Delivery,” Mediators of Inflammation, V. 2016, 2016, pp. 1-13 and Reinhard, K., Rengstl, B., Oehm, P., et al. “An RNA vaccine drives expansion and efficacy of claudin-CAR-T cells against solid tumors,” Science, V. 367, No. 6476, 2020, pp. 446-53., which are incorporated herein for reference.

When “an immunologically effective amount,” “an anti-tumor effective amount,” “a tumor-inhibiting effective amount,” or “a therapeutically effective amount” is indicated, the precise amount of the compositions of the present disclosure to be administered can be determined by a physician with consideration of individual differences in age, weight, tumor size, the extent of infection or metastasis, and condition of the patient (subject). It can be stated that a pharmaceutical composition comprising the modified cells described herein may be administered at a dosage of 10⁴ to 10⁹ cells/kg body weight, preferably 10⁵ to 10⁶ cells/kg body weight, including all integer values within those ranges. Modified cell compositions may also be administered multiple times at these dosages. The cells can be administered using infusion techniques commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly. In certain embodiments, it may be desired to administer activated T cells to a subject and then subsequently redraw the blood (or have apheresis performed), collect the activated and expanded T cells, and reinfuse the patient with these activated and expanded T cells. This process can be carried out multiple times every few weeks. In certain embodiments, T cells can be activated from 10 cc to 400 cc in blood draws. In certain embodiments, T cells are activated from blood draws of 20 cc, 30 cc, 40 cc, 50 cc, 60 cc, 70 cc, 80 cc, 90 cc, or 100 cc. Not to be bound by theory, using this multiple blood draw/multiple reinfusion protocols may select out certain populations of T cells.

The administration of the pharmaceutical compositions described herein may be carried out in any convenient manner, including by aerosol inhalation, injection, ingestion, transfusion, implantation, or transplantation. The compositions described herein may be administered to a patient subcutaneously, intradermally, intratumorally, intranodally, intramedullary, intramuscularly, by intravenous (i. v.) injection, or intraperitoneally. In embodiments, the modified cell compositions described herein are administered to subjects by intradermal or subcutaneous injection. In embodiments, the T cell compositions of the present disclosure are administered by i.v. injection. The compositions of modified cells may be injected directly into a tumor, lymph node, or site of infection. In embodiments, cells activated and expanded using the methods described herein, or other methods known in the art where T cells are expanded to therapeutic levels, are administered to patients in conjunction with (e.g., before, simultaneously, or following) any number of relevant treatment modalities, for example as a combination therapy, including but not limited to treatment with agents for antiviral therapy, cidofovir, and interleukin-2, Cytarabine (also known as ARA-C); or natalizumab treatment for MS patients; or efalizumab treatment for psoriasis patients or other treatments for PML patients. In further embodiments, the T cells described herein can be used in combination with chemotherapy, radiation, immunosuppressive agents, such as cyclosporin, azathioprine, methotrexate, mycophenolate, and FK506, antibodies, or other immunoablative agents such as CAM PATH, anti-CD3 antibodies, or other antibody therapies, cytoxin, fludaribine, cyclosporin, FK506, rapamycin, mycophenolic acid, steroids, FR901228, cytokines, and irradiation. These drugs inhibit either the calcium dependent phosphatase calcineurin (cyclosporine and FK506) or inhibit the p70S6 kinase that is important for growth factor induced signaling (rapamycin). (Liu et al., Cell 66:807-815, 1991; Henderson et al., Immun 73:316-321, 1991; Bierer et al., Curr. Opin. Immun 5:763-773, 1993; Isoniemi (supra)). In embodiments, the cell compositions described herein are administered to a subject in conjunction with (e.g., before, simultaneously, or following) bone marrow transplantation, T cell ablative therapy using either chemotherapy agents such as fludarabine, external-beam radiation therapy (XRT), cyclophosphamide, or antibodies such as OKT3 or CAMPATH. In embodiments, the cell compositions described herein are administered following B-cell ablative therapy. For example, agents that react with CD20, e.g., Rituxan, may be administered to patients. In embodiments, subjects may undergo standard treatment with high-dose chemotherapy followed by peripheral blood stem cell transplantation. In certain embodiments, following the transplant, subjects receive an infusion of the expanded immune cells of the present disclosure. In embodiments, expanded cells are administered before or following surgery. The dosage of the above treatments to be administered to a subject in need will vary with the precise nature of the condition being treated and the treatment recipient. The scaling of dosages for human administration can be performed according to art-accepted practices by a physician depending on various factors. Additional information on the methods of cancer treatment using modified cells is provided in U.S. Pat. No. 8,906,682, incorporated by reference in its entirety.

In embodiments, the method may further comprise administering an additional composition comprising CAR T cells targeting an antigen of WBCs (e.g., CD19 and BCMA).

Embodiments relate to a method of mitigating side effects in CAR T cell therapy, wherein an effective dosage of CAR T cells, which comprise a CAR that binds to a solid tumor antigen, is administered to a subject. Side effects such as diarrhea, neurotoxicity, or CRS are monitored in the subject post-administration. When any of these side effects reach a predetermined severity, an effective dosage of dasatinib is administered. If the severity of these side effects persists or increases beyond the predetermined level within a specific time frame, the dasatinib dosage is increased to a higher predetermined level until the severity of these side effects decreases to below the predetermined level. Dasatinib refers to a tyrosine kinase inhibitor used in cancer treatment. The term “effective amount” or “predetermined amount” in relation to dasatinib might need further definition, specifying that it refers to an amount sufficient to reduce the severity of a specified side effect without substantially affecting the efficacy of the CAR T cell therapy.

In embodiments, the method is used to mitigate diarrhea as a side effect of CAR T cell therapy in treating colorectal cancer. An effective dosage of a mixed CAR T cell population, comprising CAR T cells with a CAR that binds a solid tumor antigen and CAR T cells with a CAR binding CD19, is administered to a patient. The patient's diarrhea level is assessed post-administration. A first specified dosage of dasatinib is administered when the level of diarrhea reaches a predetermined severity, such as grade 2. The dasatinib dosage is increased to a second specified amount if the diarrhea level persists or escalates beyond the predetermined level within a certain time frame until the diarrhea level decreases below the predetermined level. The mixed CAR T cells comprise a population of CAR T cells that includes at least two types of CAR T cells, each type targeting a different antigen.

Embodiments relate to a method for enhancing cancer treatment. The method includes preparing a mixed CAR T cell population comprising first CAR T cells engineered to express a CAR and second CAR T cells engineered to express the CAR and a gene encoding a therapeutic agent. Tumor cells expressing multiple antigens or epitopes are contacted with the mixed CAR T cell population, and the CAR binds to one of the multiple antigens or epitopes. The mixed CAR T cell population induces a T cell response against the tumor cells, which is greater than the T cell response caused by the first CAR T cells alone.

Embodiments relate to a method of managing the side effects associated with CAR T cell therapy, particularly diarrhea. Diarrhea may be measured by the frequency and/or volume of bowel movements and increase significantly in patients receiving CAR T cell therapy. The term “predetermined level” could be clarified as a specific grade of diarrhea severity, according to a recognized grading system, such as the Common Terminology Criteria for Adverse Events (CTCAE).

The method utilizes dasatinib as an agent to control the severity of diarrhea without significantly reducing the therapeutic efficacy of CAR T cells targeting a specific solid tumor antigen, such as PAP.

In embodiments, the method employs a specific sequential treatment protocol. When the predetermined side effects level is not reduced within a predetermined timeframe after the administration of dasatinib, the dose of dasatinib is increased, and the condition of the patient is reassessed.

Embodiments relate to a method of reducing diarrhea in a subject undergoing CAR T cell therapy. This method includes administering a mixture of CAR T cells comprising both CD19-targeting CAR T cells and solid tumor-targeting CAR T cells to the subject, monitoring the level of diarrhea following administration, and administering an initial dose of dasatinib when diarrhea reaches a specified severity. If diarrhea continues to persist or worsens within a set time frame, an increased dose of dasatinib is administered until the severity of diarrhea reduces to below the specified level.

Embodiments relate to a method for reducing side effects in CAR T cell therapy with a minimal impact on the efficacy of CAR T cells targeting solid tumor antigens such as PAP. This method involves the use of dasatinib as an agent, the dose of which is modulated based on the severity of side effects observed.

Embodiments relate to a method for method for managing neurotoxicity as a side effect of CAR T cell therapy. This method includes immediate treatment with dasatinib when patients reach grade II neurotoxicity, with the dose being increased and additional treatments being introduced if the neurotoxicity progresses to higher grades despite the initial treatment. Neurotoxicity refers to a toxic effect of substances on the nervous system. The term “grade II neurotoxicity” can be clarified according to a recognized grading system like the CTCAE.

Embodiments relate to a method for a method for controlling cytokine release syndrome (CRS) as a side effect of CAR T cell therapy. This involves administering tocilizumab to a patient experiencing grade 2 fever that doesn't subside within 6 hours with conventional antipyretic measures. If fever persists post-tocilizumab administration, corticosteroids are introduced. Dasatinib can be considered for use if no improvement is seen after this step. CRS refers to a systemic inflammatory response that can be triggered by certain drugs or infections. It could be clarified that “grade 2 fever” refers to a specific fever grade according to a recognized system. Tocilizumab is an immunosuppressive drug, mainly used for treating rheumatoid arthritis (RA) and systemic juvenile idiopathic arthritis, a severe form of arthritis in children. Corticosteroids are a class of steroid hormones that can reduce inflammation and suppress the immune system.

Embodiments relate to a method for a method of enhancing the efficacy of cancer treatment by using a mixture of CAR T cells that target multiple tumor antigens. This mix includes first CAR T cells that express a CAR targeting one antigen and second CAR T cells expressing a CAR and a gene encoding a therapeutic agent.

Embodiments relate to a method for a method for managing infections that may arise during CAR T cell therapy. This involves monitoring patients for potential infections, particularly at higher CAR T cell dosages, and adjusting antibiotic regimens along with continued dasatinib administration until the side effects subside.

The present disclosure is further described by referencing the following exemplary embodiments and examples. These exemplary embodiments and examples are provided for purposes of illustration only and are not intended to be limiting unless otherwise specified. Thus, the present disclosure should in no way be construed as being limited to the following exemplary embodiments and examples, but rather, should be construed to encompass any and all variations which become evident as a result of the teaching provided herein.

### EXEMPLARY EMBODIMENTS

The following are exemplary embodiments:
1. A method of reducing side effects caused by CAR T cells when treating a subject with colorectal cancer, the method comprising: administering to the subject an effective amount of mixed CAR T cells comprising first CAR T cells comprising a chimeric antigen receptor (CAR) binding a solid tumor antigen and second CAR T cells comprising a CAR binding CD19; measuring a level of diarrhea of the subject after administering the mixed CAR T cells; administering a first predetermined amount of dasatinib when the level of diarrhea reaches a predetermined level (e.g., level 2); and administering a second predetermined amount of dasatinib when the level of diarrhea remains or is above the predetermined level within a predetermined time (e.g., 48 hours), the second predetermined amount is greater than the first predetermined amount until the level of diarrhea of the subject is less than the predetermined level.
2. A method of reducing side effects of cell therapy, the method comprising: administering an effective amount of a population of CAR T cells comprising a chimeric antigen receptor (CAR) to a subject having a tumor comprising a solid tumor antigen, the CAR binding the solid tumor antigen; measuring one or more side effects after administering the population of CAR T cells, the one or more side effects comprising at least one of diarrhea, neurotoxicity, and cytokine release syndrome (CRS); administering an effective amount of dasatinib when a level of the one or more side effects reaches a predetermined level; and administering an additional predetermined amount of dasatinib when the level of the one or more side effects remains or is above the predetermined level within a predetermined time (e.g., 48 hours), the additional predetermined amount is greater than the predetermined amount until the level of the side effect(s) of the subject is less than the predetermined level.
3. A method of enhancing cancer treatment, the method comprising: preparing mixed CAR T cells comprising first CAR T cells engineered to express a CAR and second CAR T cells engineered to express the CAR and a polynucleotide encoding a therapeutic agent; contacting tumor cells with the mixed CAR T cells, the tumor cells comprising multiple tumor antigens or epitopes, the CAR binding a tumor antigen or epitope of the multiple tumor antigens or epitopes; allowing T cell response caused by the mixed CAR T cells on the tumor cells, wherein the T cell response is greater than T cell response caused by the first CAR T cells without the second CAR T cells.
4. The method of Embodiment 3, wherein the multiple tumor antigens comprise another tumor antigen or epitope that the CAR does not bind, and/or the T cell response is measured based on a level of cytokine release, a level of anti-tumor activity, and/or a level of expansion of CAR T cells.
5. The method of Embodiment 3, wherein the therapeutic agent is IL-12.
6. The method of Embodiment 3, wherein the therapeutic agent is interferon gamma (IFNγ).
7. The method of Embodiment 3, wherein expression of the polynucleotide is regulated by an NFAT promoter.
8. The method of Embodiment 3, wherein the mixed CAR T cells comprise, at least, two of: a CAR T cell engineered to express the CAR, a CAR T cell engineered to express the CAR and IL-12, a CAR T cell engineered to express the CAR and IFNγ, and a CAR T cell engineered to express the CAR, IL-12, and IFNγ.
9. The method of Embodiment 3, wherein the CAR comprises a co-stimulatory domain of CD28.
10. A method of treating a subject having cancer cells expressing a solid tumor antigen and/or enhancing treatment of the subject, comprising the steps described in any of Embodiments 2-9.
11. The method of any of Embodiments 1-10, wherein the therapeutic agent comprises a fusion protein (e.g., polyspecific antibody) comprising: a first antigen binding domain targeting a receptor of a first immune cell; a second antigen binding domain targeting a receptor of a second immune cell; and a third antigen binding domain targeting a tumor antigen.
12. The method of any of Embodiments 1-10, wherein the therapeutic agent comprises a first fusion protein (e.g., polyspecific antibody) comprising a first antigen binding domain targeting a receptor of a first immune cell and an antigen binding domain targeting a tumor antigen; and a second fusion protein comprising a second antigen binding domain targeting a receptor of a second immune cell and an antigen binding domain targeting a tumor antigen.
13. The method of Embodiment 11 or 12, wherein the first immune cell is a T cell, and the second immune cell is a dendritic cell or macrophage.
14. The method of any of Embodiments 11-13, wherein the fusion protein is a bispecific or a trispecific antibody.
15. The method of any of Embodiments 11-14, wherein the receptor of the first immune cell and the receptor of the second immune cell are selected from receptors such as: monocyte/CD16, CD32, CD64, Mannose receptor (MR), Scavenger receptor (SR), Toll-like receptor (TLR), Phosphatidylserine receptor (PSR), CD14, CD40; NK cell/CD16, NKp46, NKp30, NKp44, NKp80, NKG2D, KIR-S, CD94/NKG2C, CRACC, Ly9, CD84, NTBA, CD3Z, 4166, CD28, 2B4; immature dendritic cell (imDC)/Complement receptor, Fc receptor (FcR), MR, TLR; mature dendritic cell (mDC)/Basic granulocyte, FcεRI, Acid granulocyte, FcεRI, Mast cells, FcεRI, FcγRIII; NKT/γδT cell; Innate lymphoid cell/Neutrophil; Dectin-1, Mac-1, TREM-1, TLR1, TLR2, TLR3, TLR4, TLR5, TLR6, TLR7, TLR8, TLR9, TLR10, NOD1, NOD2, CR4, CR1 (CD35), FcγR; T cell/CD3, CD28, 41BB, and OX40.
16. The method of any of Embodiments 11-15, wherein the fusion protein further comprises a therapeutic agent (such as a cytokine).
17. The method of Embodiment 16, wherein the therapeutic agent comprises or is a cytokine or chemotherapy agent.
18. The method of Embodiment 17, wherein the cytokine comprises or is at least one of interleukin-12 (IL-12), IL-6, and interferon gamma (IFNγ).
19. The method of any of Embodiments 11-18, wherein the first antigen binding domain comprises an agonistic antibody corresponding to the receptor of the first immune cell, and/or the second antigen binding domain comprises an agonistic antibody corresponding to the receptor of the second immune cell.
20. The method of any of Embodiments 11-19, wherein the solid tumor antigen is a non-essential tissue antigen.
21. The method of any of Embodiments 1-20, wherein the expression of the therapeutic agent is implemented by introducing a nucleic acid sequence encoding the therapeutic agent and/or the CAR, which is present in the modified cell in a recombinant DNA construct, in an mRNA, or in a viral vector.
22. The method of Embodiment 21, wherein the nucleic acid sequence is an mRNA, which is not integrated into the genome of the modified cell.
23. The method of Embodiment 21, wherein the nucleic acid sequence is associated with an oxygen-sensitive polypeptide domain.
24. The method of Embodiment 23, wherein the oxygen-sensitive polypeptide domain comprises a hypoxia inducible factor (HIF) von Hippel-Lindau (VHL) binding domain.
25. The method of Embodiment 21, wherein the nucleic acid sequence is regulated by a promoter comprising a binding site for a transcription modulator that modulates the expression and/or secretion of the therapeutic agent in the cell.
26. The method of Embodiment 25, wherein the transcription modulator is or includes Hif1a, nuclear factor of activated T cells (NFAT), forkhead box P3 (FOXP3), and/or nuclear factor kappa-light-chain-enhancer of activated B cells (NFkB).
27. The method of any of Embodiments 1-26, wherein the CAR comprises an antigen-binding domain, a transmembrane domain, and an intracellular signaling domain.
28. The method of Embodiment 27, wherein the antigen-binding domain binds to a tumor antigen selected from: TSHR, CD19, CD123, CD22, CD30, CD171, CS-1, CLL-1, CD33, EGFRvIII, GD2, GD3, BCMA, Tn antigen (Ag), PSMA, ROR1, FLT3, FAP, TAG72, CD38, CD44v6, carcinoembryonic antigen (CEA), EPCAM, B7H3, KIT, IL-13Ra2, Mesothelin, IL-11Ra, PSCA, PRSS21, VEGFR2, LewisY, CD24, PDGFR-beta, SSEA-4, CD20, Folate receptor alpha, ERBB2 (Her2/neu), MUC1, EGFR, NCAM, Prostase, prostatic acid phosphatase (PAP), ETS-related gene 2 (ELF2M), Ephrin B2, IGF-I receptor, CAIX, LMP2, glycoprotein 100 (gp100), bcr-abl, tyrosinase, EphA2, Fucosyl GM1, sLe, GM3, TGSS, HMWMAA, o-acetyl-GD2, Folate receptor beta, TEM1/CD248, TEM7R, claudin 6 (CLDN6), GPRCSD, CXORF61, CD97, CD179a, anaplastic lymphoma receptor tyrosine kinase (ALK), Polysialic acid, PLAC1, GloboH, NY-BR-1, UPK2, HAVCR1, adrenoceptor beta 3 (ADRB3), PANX3, GPR20, LY6K, OR51E2, TARP, Wilms tumor 1 (WT1), cancer/testis antigen 1 (NY-ESO-1), LAGE-1a, melanoma antigen family A 1 (MAGE-A1), legumain, human papillomavirus (HPV) E6, E7, MAGE A1, ETV6-AML, sperm protein 17, XAGE1, Tie 2, MAD-CT-1, MAD-CT-2, Fos-related antigen 1, p53, mutant p53, prostein, survivin and telomerase, galectin 8 (PCTA-1), MelanA/MART1, mutant Ras, human telomerase reverse transcriptase (hTERT), sarcoma translocation breakpoints, melanoma-associated antigen (ML-IAP), ERG (ETS-related gene), NA17, paired box 3 (PAX3), androgen receptor, cyclin B1, MYCN, RhoC, tyrosinase-related protein 2 (TRP-2), cytochrome P450 1B1 (CYP1B1), brother of regulator of imprinted sites (BORIS), cancer/testis antigen family 45 member 3 (SART3), PAXS, cancer/testis antigen (OY-TES1), lymphocyte protein tyrosine kinase (LCK), adenylate kinase 4 (AKAP-4), synovial sarcoma X breakpoint 2 (SSX2), receptor for advanced glycation end-products (RAGE-1), telomerase reverse transcriptase (hTERT), RU1, RU2, intestinal carboxyl esterase, mutant heat shock 70 kDa protein 2 (mut hsp70-2), CD79a, CD79b, CD72, leukocyte-associated immunoglobulin like receptor 1 (LAIR1), Fc fragment of IgA receptor (FCAR), leukocyte immunoglobulin like receptor A2 (LILRA2), C-type lectin domain family 12 member A (CLEC12A), bone marrow stromal cell antigen 2 (BST2), EGF like module containing, mucin like, hormone receptor like 2 (EMR2), lymphocyte antigen 75 (LY75), glypican 3 (GPC3), Fc receptor like 5 (FCRL5), and immunoglobulin lambda like polypeptide 1 (IGLL1).
29. The method of any one of Embodiments 27 and 28, wherein the intracellular signaling domain comprises a co-stimulatory signaling domain, or a primary signaling domain and a co-stimulatory signaling domain, wherein the co-stimulatory signaling domain comprises a functional signaling domain of a protein selected from: CD27, CD28, 4-166 (CD137), OX40, CD30, CD40, PD-1, inducible T cell costimulator (ICOS), lymphocyte function-associated antigen-1 (LFA-1), CD2, CD7, LIGHT, NKG2C, B7-H3, a ligand that specifically binds with CD83, CDS, ICAM-1, glucocorticoid induced tumor necrosis factor receptor (GITR), B cell activating factor receptor (BAFFR), herpes virus entry mediator (HVEM, LIGHTR), signaling lymphocytic activation molecule F7 (SLAMF7), natural killer cell receptor protein 80 (NKp80, KLRF1), CD160, CD19, CD4, CD8alpha, CD8beta, interleukin 2 receptor (IL2R) beta, IL2R gamma, IL7R alpha, integrin alpha 4 (ITGA4), very late antigen 1 (VLA1), CD49a, ITGA4, IA4, CD49D, ITGA6, VLA-6, CD49f, ITGAD, CD11d, ITGAE, CD103, ITGAL, CD11a, lymphocyte function-associated antigen 1 (LFA-1), ITGAM, CD11 b, ITGAX, CD11c, ITGB1, CD29, ITGB2, CD18, LFA-1, ITGB7, tumor necrosis factor receptor 2 (TNFR2), TNF-related activation-induced cytokine (TRANCE) or receptor activator of nuclear factor kappa-B ligand (RANKL), DNAX accessory molecule 1 (DNAM1, CD226), signaling lymphocytic activation molecule family member 4 (SLAMF4, CD244, 2B4), CD84, CD96 (Tactile), carcinoembryonic antigen-related cell adhesion molecule 1 (CEACAM1), CRTAM, Ly9 (CD229), CD160 (BY55), P-selectin glycoprotein ligand 1 (PSGL1), CD100 (SEMA4D), CD69, SLAM family member 6 (SLAMF6, NTB-A, Ly108), signaling lymphocytic activation molecule (SLAM, SLAMF1, CD150, IPO-3), B lymphocyte activator macrophage expressed (BLAME, SLAMF8), selectin P ligand (SELPLG, CD162), lymphotoxin beta receptor (LTBR), linker for activation of T cells (LAT), GRB2 related adaptor protein (GADS), lymphocyte cytosolic protein 2 (SLP-76), phosphoprotein associated with glycosphingolipid-enriched microdomains (PAG/Cbp), natural killer cell receptor 44 (NKp44), NKp30, NKp46, and natural killer group 2 member D (NKG2D).
30. The method of any one of Embodiments 1-29, wherein the CAR is replaced by a modified T cell receptor (TCR).
31. The method of Embodiment 30, wherein the TCR is derived from spontaneously occurring tumor-specific T cells in patients.
32. The method of Embodiment 30, wherein the TCR binds to a tumor antigen.
33. The method of Embodiment 32, wherein the tumor antigen comprises carcinoembryonic antigen (CEA), glycoprotein 100 (gp100), melanoma-associated antigen recognized by T cells 1 (MART-1), p53, melanoma antigen family A 3 (MAGE-A3), or cancer/testis antigen 1 (NY-ESO-1).
34. The method of Embodiment 30, wherein the TCR comprises TCRγ and TCRδ chains or TCRα and TCRβ chains, or a combination thereof.
35. The method of any of Embodiments 1-34, wherein the T cell is replaced by a natural killer (NK) cell.
36. The method of any of Embodiments 1-35, wherein the cells comprise a nucleic acid sequence encoding a binding molecule and a dominant negative form of an inhibitory immune checkpoint molecule or a receptor thereof.
37. The method of Embodiment 36, wherein the inhibitory immune checkpoint molecule is selected from: programmed death 1 (PD-1), cytotoxic T lymphocyte antigen-4 (CTLA-4), B- and T-lymphocyte attenuator (BTLA), T cell immunoglobulin mucin-3 (TIM-3), lymphocyte-activation protein 3 (LAG-3), T cell immunoreceptor with Ig and ITIM domains (TIGIT), leukocyte-associated immunoglobulin-like receptor 1 (LAIR1), natural killer cell receptor 2B4 (2B4), and CD160.
38. The method of Embodiment 37, wherein the inhibitory immune checkpoint molecule is selected from the group consisting of programmed death 1 (PD-1), cytotoxic T lymphocyte antigen-4 (CTLA-4), B- and T-lymphocyte attenuator (BTLA), T cell immunoglobulin mucin-3 (TIM-3), lymphocyte-activation protein 3 (LAG-3), T cell immunoreceptor with Ig and ITIM domains (TIGIT), leukocyte-associated immunoglobulin-like receptor 1 (LAIRI), natural killer cell receptor 2B4 (2B4), and CD 160.
39. The method of Embodiment 37, wherein the modified PD-1 lacks a functional PD-1 intracellular domain for PD-1 signal transduction, interferes with a pathway between PD-1 of a human T cell of the human cells and PD-L1 of a certain cell, comprises or is a PD-1 extracellular domain or a PD-1 transmembrane domain, or a combination thereof, or a modified PD-1 intracellular domain comprising a substitution or deletion as compared to a wild-type PD-1 intracellular domain, or comprises or is a soluble receptor comprising a PD-1 extracellular domain that binds to PD-L1 of a certain cell.
40. The method of any of Embodiments 1-39, wherein the modified cell has a reduced expression of endogenous T cell receptor alpha constant (TRAC) gene.
41. The method of any of Embodiments 1-40, wherein the modified cell comprises a first chimeric antigen receptor (CAR) binding a white blood cell antigen and a second CAR binding a solid tumor antigen.
42. The method of any of Embodiments 1-40, wherein the modified cell comprises a bispecific CAR binding a white blood cell antigen and a solid tumor antigen.
43. The method of any of Embodiments 1-42, further comprising: administering an effective amount of additional modified cell binding white blood cell antigen.
44. The method of Embodiment 43, wherein the white blood cell antigen is CD19, CD22, CD20, B cell maturation antigen (BCMA), CD5, CD7, CD2, CD16, CD56, CD30, CD14, CD68, CD11 b, CD18, CD169, CD1c, CD33, CD38, CD138, or CD13.
45. The method of Embodiment 44, wherein the white blood cell antigen is CD19, CD20, CD22, or BCMA.
46. The method of any of Embodiments 1-45, wherein the therapeutic agent comprises or is CCR2, CCR4, CXCR3, CCR6, intercellular adhesion molecule 3 (ICAM3), CCR7, lymphocyte function-associated antigen 3 (LFA-3), CCR1, CCR3, or CCR5.

### EXAMPLES

Lentiviral vectors that encode individual CAR molecules were generated and transfected with T cells, as explained below. Techniques related to cell cultures and cytotoxic T lymphocyte assay can be found in “Control of large, established tumor xenografts with genetically retargeted human T cells containing CD28 and CD137 domains,” PNAS, Mar. 3, 2009, vol. 106 no. 9, 3360-3365 and “Chimeric Receptors Containing CD137 Signal Transduction Domains Mediate Enhanced Survival of T Cells and Increased Antileukemic Efficacy In Vivo,” Molecular Therapy, August 2009, vol. 17 no. 8, 1453-1464, which are incorporated herein by reference in their entirety.

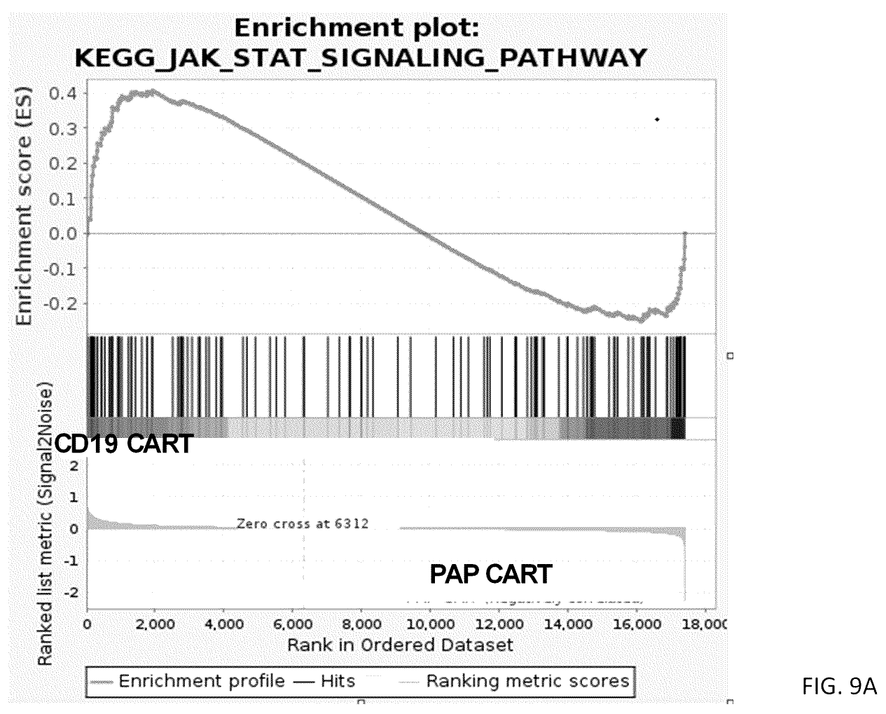

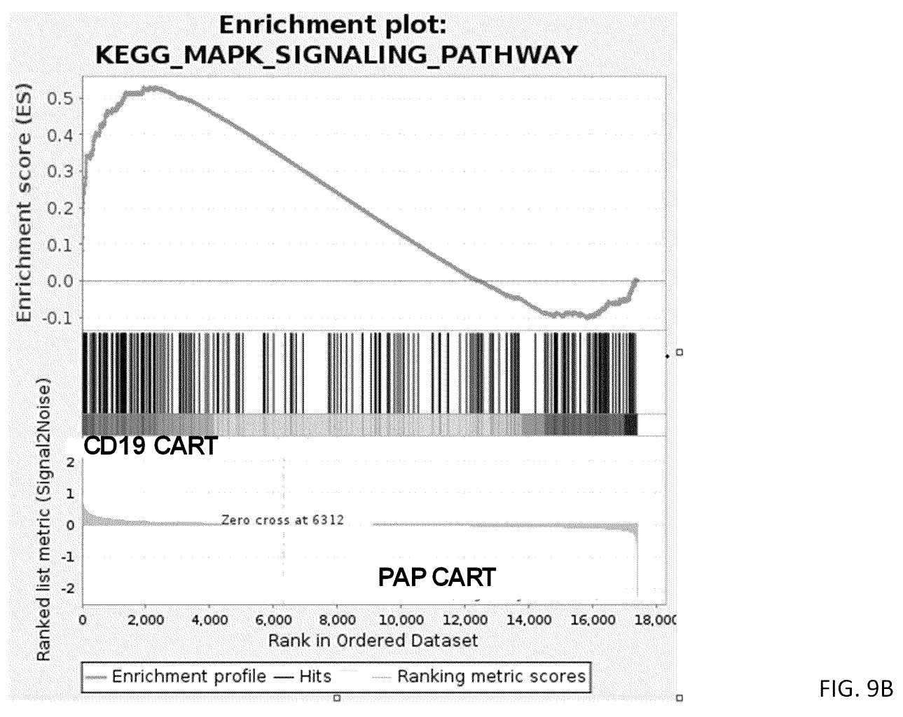

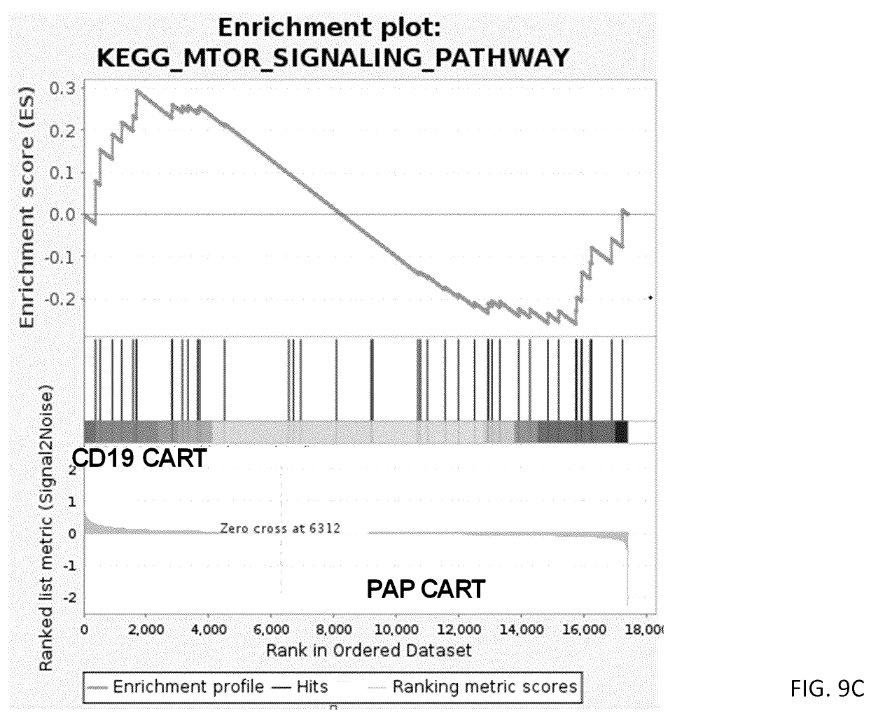

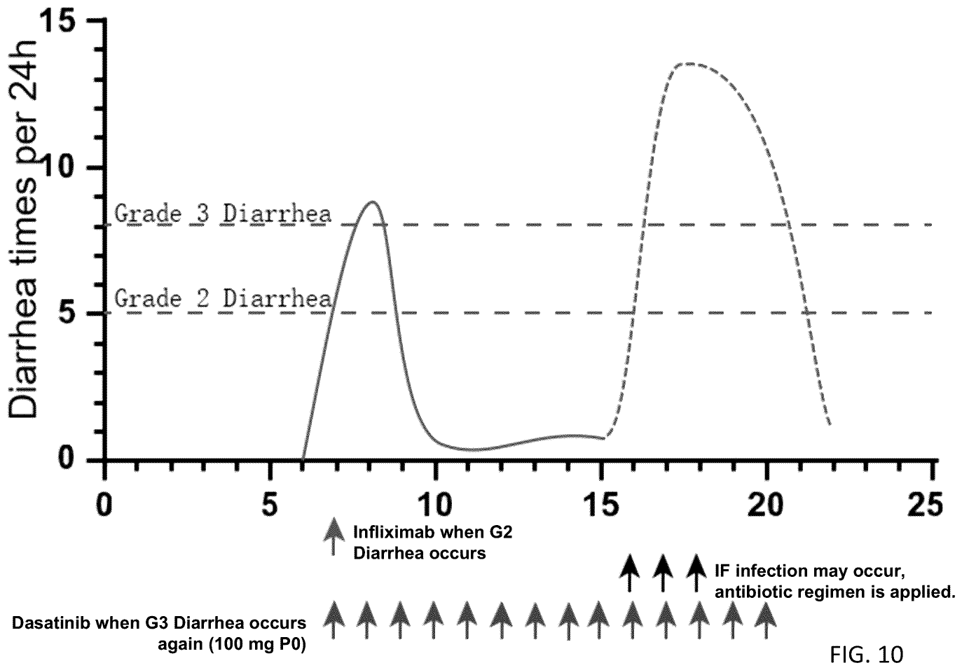

FIGS. 9A-9C illustrate alterations in the signaling pathways of CAR T cells in the CoupledCAR® system. An in vitro system was developed by co-culturing PAP CAR T cells, CD19 CAR T cells, and B cells for a 4-hour period. Analysis of the CAR T cells' signaling pathways revealed that the CD19 CAR T cells had a more enriched presence in three specific pathways: MAPK, mTOR, and JAK/STAT, when compared to solid tumor CAR T cells, such as PAP (prostatic acid phosphatase). These results align with a clinical study in which dasatinib, a tyrosine kinase inhibitor, significantly reduced CD19 CAR T cell expansion, while PAP CAR T cells were not greatly affected.

Details and associated clinical data of the CoupleCAR® technology are described in several patent publications, including PCT Publication Numbers: WO2020146743 and WO2020106843, as well as US Patent Publication Numbers: US20210060069, US20210100841, and US20220249557. These publications are incorporated by reference in their entirety.

This system was implemented in a clinical trial involving 35 colorectal cancer (CRC) patients, which were treated with mixed population of cells including GCC CAR T cells. Diarrhea, a common side effect, typically presented as early as day 7 (median=9, range 7-15) at a grade two severity or higher. The primary treatment for this was immediate administration of infliximab and, in some cases, dasatinib.

For the 1×10⁶ CAR T/kg of body weight dose, half of the patients managed their diarrhea through supportive care and antidiarrheal medication. Around 85% experienced grade 2 or higher diarrhea, 67% of whom responded well to a single dose of infliximab. At a higher dose of 2×10⁶ CAR T/kg bodyweight, only 13% of patients could control diarrhea with supportive care or antidiarrheal drugs, and all patients experienced a grade 2 or higher diarrhea, but only 43% responded to a single infliximab dose. At a even higher dose of 3×10⁶ CAR T/kg body weight, all patients experienced grade 2 or higher diarrhea, and fewer patients benefitted from a single dose of infliximab. In such cases, dasatinib was administered at 100 mg/day for over ten consecutive days until the patient's side effects reduced to grade 1 or less, and the patient was ready for discharge. As shown in Table 2, the clinical data showed that dasatinib did not appear to impact GCC CAR T cells' efficacy.

To reduce these side effects without hampering efficacy, several strategies were investigated. For instance, if a patient does not recover from grade 2 diarrhea within 48 hours, the dasatinib dosage can be increased to 90 mg twice daily until diarrhea returns to baseline. Concurrently, 1 to 2 mg/kg/day of methylprednisolone or equivalent of dexamethasone can be administered. For the 1×10⁶ CAR− T/kg dose, a 100 mg/day dosage of dasatinib can be administered, which should immediately control the diarrhea of all the patients. For the 2×10⁶ CAR T/kg dose, a 100 mg/day dosage of dasatinib can be administered to control the diarrhea of most patients; however, if no substantial improvement is seen within two days, the dosage can be increased to 90 mg twice daily. For the 3×10⁶ CAR T/kg dose, a 100 mg/day dosage of dasatinib can e administered to control the diarrhea of most patients. If there's no significant improvement within two days, the dose can be increased to 90 mg twice daily.

After the aforementioned treatments, if no relief is observed within 4 days, or if there is a subsequent recurrence of grade 2 or higher diarrhea, the patient could be experiencing an infection. Continuation of dasatinib until complete resolution of diarrhea and adjustments to the antibiotic regimen are recommended. At the 1×10⁶ CAR T/kg dose, there's a negligible or very low likelihood of infection. The same applies for the 2×10⁶ CAR T/kg dose. However, at the 3×10⁶ CAR T/kg dose, the risk of infection may be low but still noteworthy.

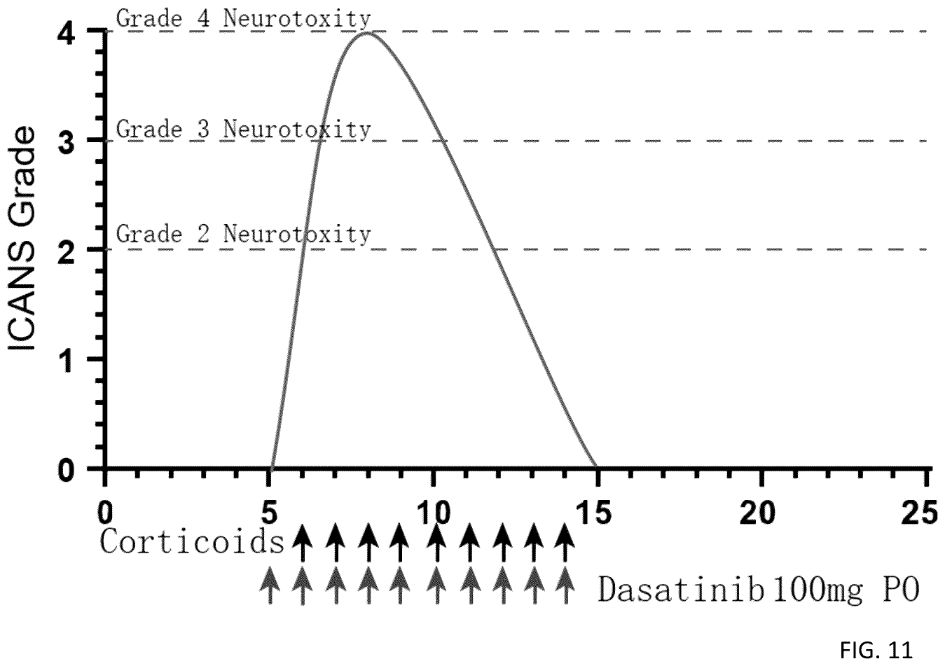

As depicted in FIG. 11, when patients exhibit a grade II neurotoxicity, a condition known as immune effector cell-associated neurotoxicity syndrome (ICANS), immediate initiation of dasatinib treatment starting with administering a dosage of 100 mg daily until the symptoms of ICANS completely subside. If, despite this treatment, the patient progresses to grade 3 neurotoxicity, the dasatinib dosage can be increased to 90 mg every 12 hours. The patient can be transferred to the ICU, and additional drugs, such as glucocorticoids, can be administered following the standard guidelines until neurotoxic symptoms are completely alleviated. If the patient progresses to grade 4 neurotoxicity under this treatment, mannitol can be added according to the guidelines along with the aforementioned drugs.

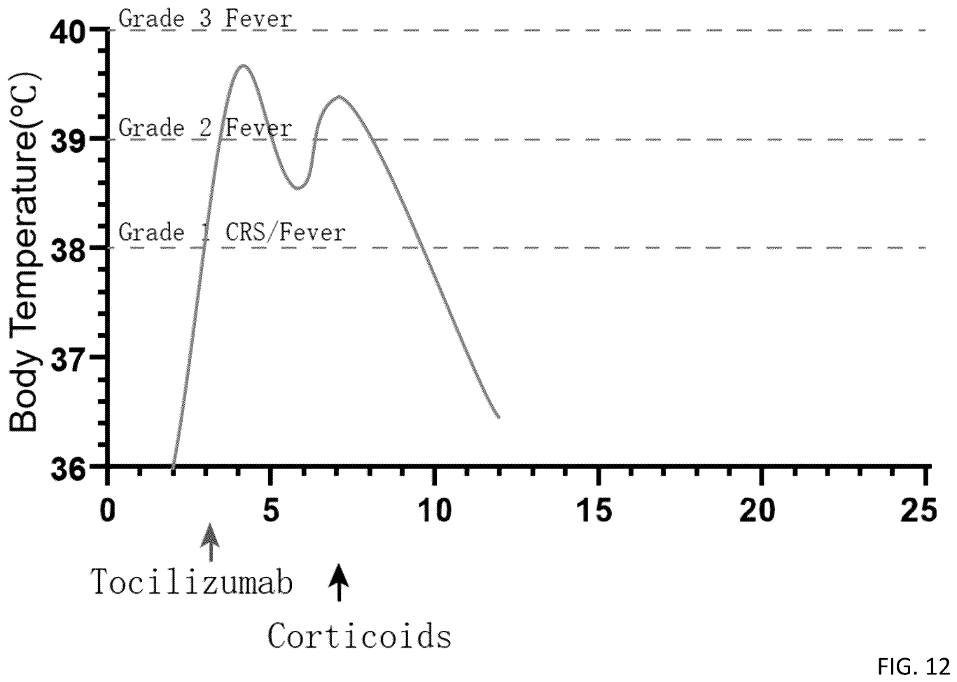

As shown in FIG. 12, for managing Cytokine Release Syndrome (CRS), tocilizumab can be administered, if the patient develops a grade 2 fever, with no signs of hypotension or hypoxia, that doesn't resolve within 6 hours using conventional antipyretic methods. This fever typically occurs earliest on day 3 (median is day 5, range is day 3-12). Tocilizumab administration results in remission in 90% of cases. However, if 24 hours after administering tocilizumab, the patient still presents with a grade 2 or higher fever or a refractory fever, treatment with corticosteroids should be initiated following ASCO guidelines. The remaining 10% of patients typically responds well to corticosteroids. If there is no improvement within 24 hours after this step, the administration of dasatinib at 100 mg once daily for 2-3 days can be administered.

TABLE 2

| | Number | Response | Disease |
|---|---|---|---|
| | of | Rate to CAR | Control |
| | Patients | T Therapy | Rate |
| 1 million cells/kg | 18 | | |
| patients who got dasatinib | 3 | 0% | 33.3% |
| patients who did not get dasatinib | 15 | 20% | 66.7% |
| 2 million+ cells/kg | 17 | | |
| patients who got dasatinib | 8 | 50% | 75% |
| patients who did not get dasatinib | 9 | 55.6% | 88.9% |

TABLE 3

| ID | SEQ |
|---|---|
| Anti-GCC scFv | SEQ ID NO: 1 |
| Anti-GCC CAR | SEQ ID NO: 2 |
| CD80 aa CAR | SEQ ID NO: 3 |
| CD86 aa CAR | SEQ ID NO: 4 |
| TNFSF9/41BBL aa CAR | SEQ ID NO: 5 |
| CD28 antibody aa//Anti-CD28 antibody | SEQ ID NO: 6 and 7 |
| scFv-(mAb 9.3) CAR (VH and VL) | |
| 41BB antibody (Anti-CD137 antibody | SEQ ID NO: 8 and 9 |
| scFv) CAR (VH and HL) | |

All publications, patents and patent applications cited in this specification are incorporated herein by reference in their entireties as if each individual publication, patent or patent application were specifically and individually indicated to be incorporated by reference. While the foregoing has been described in terms of various embodiments, the skilled artisan will appreciate that various modifications, substitutions, omissions, and changes may be made without departing from the spirit thereof.

1. A method for reducing side effects associated with CAR T cell therapy, the method comprising:
administering dasatinib to a patient undergoing CAR T cell therapy for treating solid tumor, wherein the dasatinib reduces the side effects associated with the CAR T cell therapy, and wherein the dasatinib does not inhibit the efficacy of CAR T cells targeting the solid tumor.

2. The method of claim 1, wherein the dasatinib is administered at a dosage of 100 mg per day.

3. The method of claim 1, wherein the dasatinib is administered when the patient experiences grade 2 or higher diarrhea.

4. The method of claim 3, wherein the dosage of dasatinib is increased to 90 mg twice daily if the patient does not recover from grade 2 diarrhea within 48 hours.

5. The method of claim 1, wherein the dasatinib is administered until the side effects of the patient are reduced to grade 1 or less.

6. The method of claim 1, wherein the dasatinib is administered in combination with infliximab when diarrhea is observed.

7. The method of claim 1, wherein the side effects are diarrhea, Cytokine Release Syndrome (CRS), or Immune effector cell-associated neurotoxicity syndrome (ICANS).

8. The method of claim 7, wherein the dasatinib is administered immediately upon the observation of grade 2 ICANS.

9. The method of claim 8, wherein the dasatinib dosage is increased to 90 mg twice daily if the patient progresses to grade 3 ICANS.

10. The method of claim 1, wherein the dasatinib is administered in combination with corticosteroids.

11. The method of claim 1, wherein the dasatinib is administered in combination with corticosteroids, when no improvement is observed within 24 hours following administration of tocilizumab for CRS management.

12. The method of claim 1, wherein dasatinib is administered when no improvement is observed within 24 hours following administration of corticosteroid for CRS management.

13. The method of claim 1, wherein the patient is undergoing CAR T cell therapy for the treatment of solid tumors, and optionally wherein the solid tumor is colorectal cancer (CRC).

14. The method of claim 1, wherein the patient is treated with a CAR T therapy system comprising a mixed population of CAR T cells targeting solid tumor cells, CD19 cells, and B cells.

15. The method of claim 1, further comprising administering methylprednisolone or dexamethasone along with dasatinib.

16. The method of claim 1, wherein a dose of the dasatinib is a adjusted based on based on the quantity of CAR T cells being administered to the patient.

17. A method for mitigating diarrhea as a side effect of CAR T cell therapy of colorectal cancer treatment, the method comprising:
administering an effective dose of a mixed CAR T cell population to a patient, the mixed CAR T cell population comprising CAR T cells with a CAR binding a solid tumor antigen, and CAR T cells with a CAR binding CD19;
assessing severity of the patient's diarrhea post-administration of the mixed CAR T cell population;
administering a first specified dose of dasatinib when the severity of the patient's diarrhea reaches a predetermined severity requiring treatment; and
increasing the dose of dasatinib to a second specified dose, greater than the first specified dose, when the severity of the patient's diarrhea persists or escalates beyond the predetermined severity within a specified time frame, and optionally, the specified time frame is 48 hours, and
continuing to administer the second specified dose until the severity of the patient's diarrhea is reduced below the predetermined severity.

18. A method for mitigating one or more side effects of CAR T cell therapy, the method comprising:
administering an effective dose of a mixed population of CAR T cells to a patient with a solid tumor, the CAR T cells expressing a CAR that binds a solid tumor antigen on the solid tumor;
monitoring the patient for one or more side effects post-administration of the mixed population of CAR T cells;
administering an effective dose of dasatinib when one or more of these side effects reach a predetermined severity requiring treatment; and
increasing the dosage of dasatinib to a higher predetermined dose when one or more of these side effects persist or increase beyond the predetermined severity within a specified time frame; and
continuing to administer the higher predetermined dose until the severity of these side effects is reduced below the predetermined severity.

19. The method of claim 18, wherein the specified time frame is 48 hours.

20. The method of claim 18, wherein the one or more side effects comprise diarrhea, neurotoxicity, CRS, or a combination thereof.
